(19) United States
(12) Patent Application Publication
(10) Pub. No.: US 2017/0022131 A1
(43) Pub. Date: Jan. 26, 2017

(54) ENERGY EFFICIENT ETHANOL RECOVERY BY ADSORPTION

(71) Applicant: Joule Unlimited Technologies, Inc., Bedford, MA (US)

(72) Inventor: Kevin William Hettenbach, Belmont, MA (US)

(21) Appl. No.: 15/300,891

(22) PCT Filed: Apr. 2, 2015
(86) PCT No.: PCT/US15/24019

§ 371 Date: Sep. 30, 2016

Related U.S. Application Data
(60) Provisional application No. 61/974,205, filed on Apr. 2, 2014.
(60) Provisional application No. 61/974,218, filed on Apr. 2, 2014.

(51) Int. Cl. C07C 29/76; B01D 53/08

(52) U.S. Cl. CPC C07C 29/76; B01D 53/08; B01D 2257/708

(57) ABSTRACT

A method and system for recovering a volatile organic compound from a dilute aqueous phase. The method may include separating volatile organic compound from the aqueous phase by using carrier gas to generate a solvent-laden vapor stream, feeding a solvent-laden vapor stream to a mass of carbon adsorbent and enabling the solvent to be absorbed and separated from the solvent-laden vapor stream, releasing the absorbed volatile organic compound, and condensing the released volatile organic compound to form a condensate. The system may include a vapor phase source containing ethanol, at least one carbon bed containing a mass of coconut shell carbon, a steam source in fluid communication with the carbon bed, and a condenser in fluid communication with the carbon bed. The method and system may also utilize microbeads as an absorbent and may be configured so the capacity is scalable from lab scale to production scale.

### RELATED APPLICATIONS

This application claims the benefit of U.S. Prov onal Application No. 61/974,205, filed Apr. 2, 2014, and U.S. Provisional Application No. 61/974,218, filed Apr. 2, 2014, each of which is incorporated by reference in their entirety.

### TECHNICAL FIELD

The present disclosure is directed towards an energy-efficient process for the recovery of a volatile organic compound from an aqueous phase using adsorbent media, and more particularly, recovery of ethanol from a dilute ethanol aqueous phase.

### BACKGROUND

The world's energy demands continue to increase while the supplies of non-renewable sources diminish. Concerns regarding the limited supplies, rising cost, and environmental concerns have spurred the development of alternative, renewable, and clean energy sources. The sun has been one source of clean renewable energy that for years has inspired development of various energy capturing methods.

One method of capturing the sun's energy developed by Joule Unlimited Technologies (“Joule”) referred to as HELIOCULTURE® is the use of photobioreactors, which contain microorganisms that turn sunlight, carbon dioxide, and water into biofuels. The microorganisms are engineered to directly photosynthetically convert sunlight and carbon dioxide into organic compounds, for example, ethanol (“EtOH”), which among other things, can be used as a liquid motor fuel or for blending with other fuel stocks. Ethanol blends within conventional gasoline or diesel motor fuels is increasing worldwide. Joule's photobioreactor ethanol production method does not require additional feedstock, and does not therefore burden supplies of food/feed corn, fertile agricultural land, or available potable water like traditional corn-fermentation ethanol production. These are among the many advantages of Joule's photobioreactor based ethanol production method as compared to traditional production methods. One challenge in bioreactor-based production processes is recovering and concentrating the organic compounds produced by the microorganisms. In some processes for producing volatile organic compounds, the compound produced is in a dilute aqueous stream (e.g., 0.2 wt % 6.7 wt %) that needs to be recovered from the liquid and vapor phases and purified to meet fuel grade specifications (e.g., greater than 98.7% w/w EtOH). Methods for recovering and concentrating the dilute volatile organic compounds exist (e.g., distillation, evaporation, molecular sieves, membrane filtration, liquid adsorption, etc.), but the energy input required can be exorbitant and as a result the fuel production becomes less economically viable.

For example, with regard to ethanol, for diluted solutions (e.g., 2-3 wt %) the relative volatility of water is in the range of 11-12 dependent on temperature. Relative volatility defines the upper limit of enrichment that can be obtained in one section of a distillation column without a condenser. The achievable enrichment ratio is about 10, which means that 1 wt % ethanol in an aqueous solution can be concentrated to about 10 wt % vapor distillate; 2 wt % to 20%, and so on. The energy required for stripping is normally provided by steam in either direct (i.e., live steam) or indirect mode (i.e., reboiler). The latent heat of evaporation of water is 2.2 MJ/kg, thus employing a stripper in indirect mode for primary ethanol enrichment from 2 wt % to 20 wt % results in 20 MJ/kg energy consumption per 1 kg of extracted ethanol. This energy investment constitutes 70% of the total enthalpy of ethanol combustion (i.e., 29.7 MJ/kg), which is far too high to make this an economically viable primary recovery option. Further enrichment of distillate from 20 wt % to fuel grade (i.e., greater than 98.7%) would require another 4 to 7 MJ/kg.

In consideration of the above described challenge, the present disclosure provides an energy efficient method and system for recovering a dilute volatile organic compound from an aqueous phase using adsorption.

### SUMMARY

In one aspect, the present disclosure is directed to a method for recovering a volatile organic compound from a dilute aqueous phase comprising separating the volatile organic compound from the aqueous phase by using a carrier gas to generate a solvent-laden vapor stream, feeding a solvent-laden vapor stream to a mass of carbon adsorbent and enabling the solvent to be absorbed and separated from the solvent-laden vapor stream, releasing the absorbed volatile organic compound_{;} and condensing the released volatile organic compound to form a condensate.

In another embodiment, the absorbed volatile organic compound can be released by heating the mass of carbon absorbent. In another embodiment, the absorbed volatile organic compound can be released by pressure swing adsorption. In another embodiment, the volatile organic compound can be ethanol, the dilute aqueous phase can be a photobioreactor ethanol titer, and the solvent-laden vapor stream can be an ethanol laden vapor stream. In another embodiment, the mass of carbon adsorbent can include a coconut shell carbon.

In another embodiment, the method can further comprise feeding the ethanol laden vapor stream until ethanol breakthrough, wherein ethanol breakthrough occurs more than 1 hour after starting. In another embodiment, the ethanol concentration in the laden vapor stream can be about 0.5 mol %. In another embodiment, the coconut shell carbon can have an ethanol adsorption breakthrough capacity greater than 0.2 g/g carbon. In another embodiment, the coconut shell carbon can have an ethanol to water adsorption selectivity ratio at ethanol breakthrough of greater than 5. In another embodiment, the coconut shell carbon can have an ethanol adsorption efficiency of greater than 99.6% at ethanol breakthrough.

In another embodiment, the coconut shell carbon can have a mass transfer zone of less than 6 inches. In another embodiment, the coconut shell carbon particle size can be between about 2.36 mm and 4.75 mm. In another embodiment, the coconut shell carbon can have a CTC activity of greater than 50%, an Iodine number greater than 1000 mg/g, moisture content less than 5%. In another embodiment, the ethanol feed concentration can be greater than 1 mol % and the percent ethanol adsorbed by the coconut shell carbon can be greater than 80%. In another embodiment, the ethanol concentration in the vapor phase can be less than about 0.01 mol % to about 0.8 mol %. In another embodiment, the photobioreactor ethanol titer ranges from about 0.037 wt % to about 6.7 wt %.

In another embodiment, the ethanol vapor phase can be a product of a photobioreactor ethanol production process. In another embodiment, the method can further comprise feeding the ethanol laden air stream to the mass of carbon at a temperature of about 37° C. In another embodiment, the ethanol concentration of the condensate can be at least 15 times greater than the photobioreactor ethanol titer. In another embodiment, releasing the absorbed volatile organic compound can comprise heating the mass of carbon absorbent by supplying steam to the mass of carbon absorbent at a steam loading of between about 0.17 kg steam/kg carbon to about 0.30 kg steam/kg carbon. In another embodiment, the steam regeneration energy requirement can be about 5 MJ/kg EtOH or less for at least 10 cycles, wherein the photobioreactor ethanol titer is 2 wt % and the concentration of the ethanol laden vapor stream is about 0.5 mol %. In another embodiment, an increase in the mass of carbon of at least 39X produces an equivalent ethanol breakthrough capacity and an equivalent condensate concentration based on the photobioreactor ethanol titer concentration.

In another aspect, the present disclosure can be directed to a system for recovering and concentrating ethanol from a vapor phase comprising a vapor phase source containing ethanol, at least one carbon bed containing a mass of coconut shell carbon, a steam source in fluid communication with the carbon bed, and a condenser in fluid communication with the carbon bed. In another embodiment, at least one carbon bed can be configured to receive the vapor phase enabling the ethanol to be absorbed by the mass of coconut shell carbon, the steam source can be configured to heat the mass of coconut shell carbon causing the release of the absorbed ethanol, and the condenser can be configured to cool the released ethanol forming a condensate.

In another embodiment, the system can further comprise a photobioreactor ethanol production system producing the ethanol vapor phase. In another embodiment, the photobioreactor ethanol titer is 2 wt % and ethanol vapor phase concentration can be about 0.5 mol %. In another embodiment, at least one carbon bed can be configured to receive the vapor phase containing ethanol until ethanol breakthrough, wherein ethanol breakthrough occurs more than 1 hour after starting. In another embodiment, the coconut shell carbon can have an ethanol adsorption breakthrough capacity greater than 0.2 g/g carbon.

In another embodiment, the coconut shell carbon can have an ethanol to water adsorption ratio at breakthrough of greater than 5. In another embodiment, the coconut shell carbon can have an ethanol adsorption efficiency of greater than 99.6% at ethanol breakthrough. In another embodiment, the coconut shell carbon can have a mass transfer zone of less than 6 inches. In another embodiment, the coconut shell carbon particle size can have between about 2.36 mm and 4.75 mm. In another embodiment, the coconut shell carbon can have a CTC activity of greater than 50%, an Iodine number greater than 1000 mg/g, moisture content less than 5%.

In another embodiment, the ethanol feed concentration in the vapor phase can be greater than 1 mol % and the percent ethanol adsorbed by the coconut shell carbon can be greater than 80%. In another embodiment, the ethanol concentration in the vapor phase can be less than about 0.01 mol % to about 0.8 mol %. In another embodiment, the photobioreactor ethanol production system generates an ethanol titer that can be about 0.037 wt % to about 6.7 wt %. In another embodiment, the ethanol vapor phase can be a product of a photobioreactor process. In another embodiment, the system can further comprise a heated gas source configured to feed gas to the mass of carbon to dry the carbon, wherein the temperature of the gas is from 75° C. to 80° C.

In another embodiment, the ethanol concentration of the condensate can be at least 15 times greater than the photobioreactor ethanol titer. In another embodiment, the steam source can provide a steam load of between about 0.17 kg steam/kg carbon to about 0.30 kg steam/kg carbon. In another embodiment, the steam regeneration energy requirement can be about 5 MJ/kg EtOH or less for at least 10 cycles, wherein the ethanol titer is 2 wt % and the ethanol vapor phase concentration is about 0.5 mol %.

In another aspect, the present disclosure can be directed to a method for recovering a volatile organic compound (VOC) from a VOC laden vapor stream comprising feeding the VOC laden vapor stream to an adsorber containing a falling mass of microbeads, enabling the VOC to be absorbed and separated from the VOC laden vapor stream, heating the adsorbed VOC and the falling mass of microbeads to release the VOC, and stripping and condensing the released VOC to form a condensate.

In another embodiment, the VOC and the falling mass of microbeads can be heated by indirect contact using steam. In another embodiment, each step of the method can be performed simultaneously and continuously. In another embodiment, the VOC can be ethanol and the ethanol concentration in the vapor stream can be about 0.01 mol % to about 0.8 mol %, In another embodiment, the VOC can be ethanol and the ethanol vapor stream can be a product of a photobioreactor process. In another embodiment, the method can further comprise removing the adsorbed water from the falling mass of microbeads to release and separate at least a portion of the water before releasing the adsorbed VOC.

In another embodiment, stripping can comprise feeding an inert stripper gas stream counter-flow to the falling mass of microbeads to capture the released VOC and supply it to a condenser. In another embodiment, the VOC can be ethanol and the ethanol vapor stream can be a product of a photobioreactor process, and the inert stripper gas stream used for stripping is CO₂ that is recycled to the photobioreactor process. In another embodiment, the VOC can be ethanol and the ethanol concentration of the condensate can range from about 80 wt % to about 95 wt %. In another embodiment, the VOC laden vapor stream discharged from the adsorber can be recycled back to a photobioreactor process.

In another aspect, the present disclosure is directed to a system for recovering and concentrating a volatile organic compound (VOC) from a dilute VOC vapor stream comprising a column comprising at least an adsorber, a transition, and a stripper in fluid communication. The system can further comprise a dilute VOC vapor stream in fluid communication with the adsorber, a stripper gas stream in fluid communication with the stripper, a plurality of microbeads configured to fall through the column and adsorb and desorb at least a portion of the VOC vapor, a heat source in fluid communication with the stripper, and a condenser configured to cool the desorbed VOC vapor and form a VOC condensate.

In another embodiment, wherein the VOC is ethanol the system can further comprise a photobioreactor system producing the dilute ethanol vapor stream. In another embodiment, the VOC can be ethanol and the concentration of ethanol in the dilute vapor stream can be about 0.04 mol % to about 1.8 mol %. In another embodiment, the heat source can be configured to heat the falling microbeads and adsorbed VOC vapor causing the VOC vapor to desorb, wherein the heating is done by indirect contact with the falling microbeads. In another embodiment, the system can be configured for continuous operation.

In another embodiment, the transition can be configured to remove at least a portion of the water before releasing the adsorbed VOC. In another embodiment, the falling microbeads in the stripper operate as a moving bed and the speed of the bed can correspond to the microbeads’ residence time for efficient VOC desorption. In another embodiment, the VOC can be ethanol and the dilute ethanol vapor can be a product of a photobioreactor process, and the stripper gas source is CO₂ that is recycled back to the photobioreactor process. In another embodiment, the VOC can be ethanol and the ethanol concentration of the ethanol condensate can range from about 80 wt % to about 95 wt %. In another embodiment, the VOC can be ethanol and the aqueous ethanol vapor stream discharged from the adsorber can be recycled. In another embodiment, a structured packing within the column can be configured such that the pressure drop is less than about 0.04 psi.

Additional objects and advantages of the present disclosure will be set forth in part in the description which follows, and in part will be obvious from the description, or may be learned by practice of the present disclosure. The objects and advantages of the present disclosure will be realized and attained by means of the elements and combinations particularly pointed out in the appended claims.

It is to be understood that both the foregoing general description and the following detailed description are exemplary and explanatory only and are not restrictive of the present disclosure as claimed.

The accompanying drawings, which are incorporated in and constitute a part of this specification, illustrate several embodiments of the present disclosure and together with the description, serve to explain the principles of the present disclosure.

### BRIEF DESCRIPTION OF THE DRAWINGS

FIG. 1 is a flow chart of a method for recovering a volatile organic compound from a dilute aqueous phase, according to an exemplary embodiment.

FIG. 2 is a flow diagram of an apparatus configured to recover a volatile organic compound from a dilute aqueous phase, according to an exemplary embodiment.

FIG. 3 is a flow chart of a method of adsorption mode, according to an exemplary embodiment.

FIG. 4 is a flow chart of a method of regeneration mode, according to an exemplary embodiment.

FIG. 5A is a plot of ethanol breakthrough curves, according to an exemplary embodiment.

FIG. 5B is a drawing showing the relationship of the variables used to calculate the mass transfer zone length, according to an exemplary embodiment.

FIGS. 6A, 6B, 6C, 6D, 6E, and 6F plot the ethanol and water mass spectrometer concentration profiles for the inlet and outlet of a carbon bed, according to an exemplary embodiment.

FIG. 7A plots the ethanol to water adsorption selectivity versus the ethanol vapor feed concentration, according to an exemplary embodiment.

FIG. 7B plots the ethanol adsorption capacity versus the ethanol feed vapor concentration, according to an exemplary embodiment.

FIG. 8 plots the ethanol breakthrough curves at concentrations from 0.04-1.8 mol %, according to an exemplary embodiment.

FIGS. 9A, 9B, 9C, 9D, 9E, 9F, 9G, 9H, 9I, and 9J plot the ethanol and water concentration as measured by the mass spectrometer at the inlet and outlet of the carbon bed for different ethanol concentrations, according to an exemplary embodiment.

FIG. 10A plots the ethanol condensate concentration following regeneration for 10 cycles of ambient drying and heat drying, according to an exemplary embodiment.

FIG. 10B plots steam regeneration energy for 10 cycles of ambient air drying and heated air drying, according to an exemplary embodiment.

FIG. 11 is a flow chart of a method for recovering ethanol from a dilute aqueous phase using continuous adsorption/regeneration, according to an exemplary embodiment.

FIG. 12 is a schematic drawing showing a falling microbeads reactor, according to an exemplary embodiment.

FIG. 13 is a flow diagram of a pilot scale apparatus configured to recover a volatile organic compound from a dilute aqueous phase, according to an exemplary embodiment,

FIG. 14 is a plot of ethanol adsorption temperature profiles and ethanol outlet concentration versus time.

FIG. 15 is a plot of ethanol condensate concentration and condensate mass versus time.

Reference will now be made in detail to the present embodiments of the present disclosure, examples of which are illustrated in the accompanying drawings. Wherever possible, the same reference numbers will be used throughout the drawings to refer to the same or like parts.

### DETAILED DESCRIPTION

The present disclosures are described herein with reference to illustrative embodiments for a particular application, such as, ethanol recovery and concentration from a dilute aqueous ethanol stream. It is understood that the embodiments described herein are not limited thereto. Those having ordinary skill in the art and access to the teachings provided herein will recognize additional modifications, applications, embodiments, and substitution of equivalents that all fall with the scope of the present disclosure. Accordingly, the present disclosures are not limited by the foregoing or following descriptions.

The present disclosures are described herein with reference to illustrative embodiments for a particular application, such as, ethanol recovery and concentration from a dilute aqueous ethanol stream. It is understood that the embodiments described herein are not limited thereto. Those having ordinary skill in the art and access to the teachings provided herein will recognize additional modifications, applications, embodiments, and substitution of equivalents that all fall with the scope of the present disclosure. Accordingly, the present disclosures are not limited by the foregoing or following descriptions.

Commonly-assigned U.S. Pat. No. 8,304,209 is one example of a system that enables production of volatile organic compounds (e.g., ethanol, biodie el fuel, etc.) using microorganisms that consume sunlight and carbon dioxide and secrete materials of interest, such as, volatile organic compounds (VOCs). The specific VOC produced can be selected based on the engineered microorganisms being used. The microorganisms can be continuously circulated in an aqueous stream (e.g., non-potable or potable water) by the introduction of a carbon dioxide stream. The microorganisms can secrete the VOCs into the aqueous stream, from which they can be separated.

Volatile organic compound (VOC) as used herein is a broad term, and can refer to, for example, any organic compounds that have a high vapor pressure at ordinary room temperature or any organic chemical including those whose composition makes it possible for evaporation under substantially normal atmospheric conditions of temperature and pressure. VOC as used herein can include very volatile organic compounds (VVOC) and semi volatile organic compounds (SVOC) as those terms are understood in the art.

According to an exemplary embodiment, the VOC produced can be ethanol, the concentration of ethanol in the aqueous stream can vary. For example, the range can be about 0.2 to 7.0 wt %, 0.2 to 6.0 wt %, 0.2 to 5.0 wt %, 0.2 to 4.0 wt %, 0.2 to 3.0 wt %, 0.2 to 2.0 wt %, 0.2 to 1.0 wt %, 0.2 to 0.5 wt %, 0.5 to 7.0 wt %, 1.0 to 7.0 wt %, 2.0 to 7.0 wt %, 3.0 to 7.0 wt %, 4.0 to 7.0 wt %, 5.0 to 7.0 wt %, 6.0 to 7.0 wt %, 0.5 to 6.0 wt %, 1.0 to 6.0 wt %, 1.5 to 5.0 wt %, 1.5 to 4.0 wt %, 1.5 to 3.0 wt %, 1.5 to 2.5 wt %, 1.5 to 2.0 wt %, or 2.0 to 2.5 wt %. In yet another exemplary embodiment, the concentration of ethanol in the aqueous stream can be, for example, about 0.2 wt %, 0.4 wt %, 0.6 wt %, 0.8 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt %, 3.2 wt %, 3,4 wt %, 3.6 wt %, 3.8 wt %, 4.0 wt %, 4.2 wt %, 4.4 wt %, 4.6 wt %, 4.8 wt %, 5.0 wt %, 5.5 wt %, 6.0 wt %, 6.5 wt %, or 7.0 wt %. Such concentrations are lower compared to the typical 6 to 14 wt % produced by corn and cellulosic ethanol fermentation. As a result, starting from a lower concentration would imply that more energy would be needed to recover and concentrate the ethanol to acceptable fuel grade (i.e., greater than 98.7 wt % or higher).

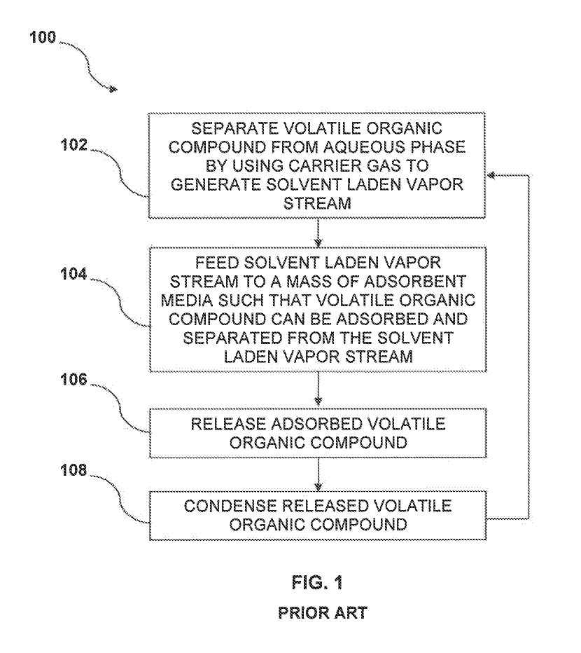

According to an exemplary process embodiment depicted in FIG. 1, a method 100 of recovering a VOC from a dilute aqueous phase is described below. The method can comprise steps 102, 104, 106, and 108 as shown in FIG. 1. Step 102 can comprise separating the VOC from the aqueous phase by using a carrier gas (e.g., air or nitrogen) to generate a solvent-laden vapor stream. Step 104 can comprise feeding the solvent-laden vapor stream to a mass of adsorbent media such that the volatile organic compound can be adsorbed and separated from the solvent-laden vapor stream. Step 106 can comprise releasing the adsorbed volatile organic compound. Step 108 can comprise condensing the released volatile organic compound. In other embodiments, the step of separating the VOC from the aqueous phase by using a carrier gas may be omitted whereby air is utilized to remove excess oxygen produced in the VOC production process which generates a solvent-laden vapor stream. For example, the head space in a sump tank or other structure in a storage, transport, or separation process may contain a solvent-laden vapor stream. In yet another example, carbon dioxide not utilized in the VOC production process can act as a carrier gas and separate the VOC from the aqueous phase.

Method 100, as depicted in FIG. 1 describes a prior art process. Method 100 was used as the foundation to start developing a more energy efficient method of recovering and concentrating VOCs from a dilute aqueous phase. Initial development focused on recovering ethanol from a dilute aqueous phase, according to an exemplary embodiment. However, the embodiments of the present disclosure are not limited to ethanol (EtoH), but rather the teachings of the present disclosure can be applied to other recovery applications for materials of interest, for example, alcohols (e.g., butanol, MeOH, propanol, isopropanol, etc.), fuels (e.g., hydrocarbons (aromatic and aliphatic), diesel, biodiesel, biofuel, gasoline, etc.), ketones, esters, chlorinated solvents, brominated solvents, fluorinated solvents, alkanes, alkenes, fatty esters, sugars, olefins, and derivatives thereof, etc.

### Carbon Adsorbent

According to one aspect of exemplary embodiments, the energy efficiency of method 100 can be improved by utilizing an adsorbent media that provides improved performance. According to an exemplary embodiment, a carbon-based adsorbent was selected. An ideal carbon adsorbent would demonstrate high ethanol recovery efficiency, high ethanol adsorption capacity, high ethanol selectivity (i.e., versus water adsorption), and increased steam regeneration efficiency.

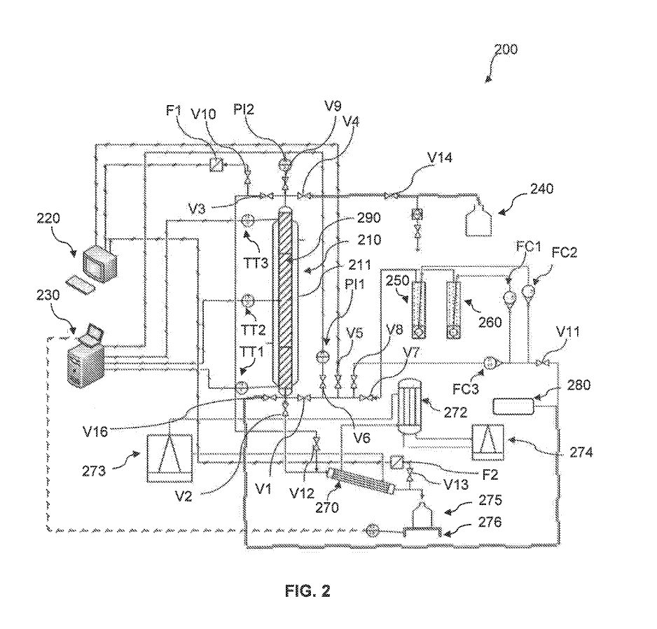

To evaluate the performance of different carbon adsorbents, an adsorption and regeneration apparatus 200 was assembled, an exemplary construction of which is shown in FIG. 2. Apparatus 200 can comprise a carbon bed 210, mass spectrometer 220, data collector 230, steam generator 240, ethanol sparger 250, water sparger 260, heat exchanger 270, and nitrogen gas source 280.

As shown in FIG. 2, apparatus 200 can be assembled such that nitrogen gas source 280 can be in fluid communication with the inlet of ethanol sparger 250 and the inlet of water sparger 260 as well as the bottom of carbon bed 210 through valves V16 and V8. The outlet of ethanol sparger 250 and the outlet of water sparger 260 can combine and be in fluid communication with the bottom of carbon bed 210 through valves V1 and V7. Between valves V1 and V7 can be branch connections to valves V5 V6, and V8. Valve V5 can be in fluid communication with mass spectrometer 220 while valve V6 can be in fluid communication with a pressure indicator PI1 configured to measure adsorption inlet pressure, which can be in electrical communication with data collector 230.

Nitrogen from nitrogen gas source 280 can be bubbled into ethanol sparger 250 and water sparger 260 at a controlled flow rate using Flow Controllers FC1 and FC2 (e.g., flow controllers available from Brooks Instruments of Hatfield, Pa.). The flow ratio of nitrogen ethanol sparger 250 and water sparger 260 can be advantageously adjusted to produce an ethanol laden vapor stream and a water vapor stream of desired ethanol inlet concentration. The relative humidity in nitrogen can also be varied. Ethanol sparger 250 and water sparger 260 can be at room temperature or they can be heated using heat plates depending on the testing parameters enabling temperature adjustment of the vapor streams.

The vapor streams produced by ethanol sparger 250 and water sparger 260 can combine and flow through valves V7 and V1 into the bottom of carbon bed 210. Carbon bed 210 can vary in diameter and length, for example, carbon bed 210 may be 1 inch in diameter by 15 inches in height, 3 inches in diameter by 10 inches in height, or 1.5 inches in diameter by 36 inches in height. In other embodiments, carbon bed 210 may be of a different size. Carbon bed 210 may be configured to receive a mass of carbon 290. The carbon capacity of carbon bed 210 may vary based on the size of the bed.

Carbon bed 210 can be formed of a variety of different metals or metals alloys, for example, stainless steel. Carbon bed 210 can be oriented vertically to optimize carbon packing density. Carbon bed 210 can further comprise a heat jacket 211 configured to heat carbon bed 210 if desired. The lines between ethanol sparger 250, water sparger 260, and carbon bed 210 can be heated using heat tape (not shown) or other means in order to avoid vapor condensation. Carbon bed 210 can further comprise temperature transmitters TT1, TT2, and TT3 in electrical communication with data controller 230 configured to detect the carbon bed 210 inlet, mid-point, and outlet temperature, respectively.

As shown in FIG. 2, mass spectrometer 220 can be in fluid communication with carbon bed 210 inlet through valve V5 and outlet through a valve V10. Between valve V10 and mass spectrometer 220 can be a particulate filter F1 configured to protect the mass spectrometer from mass of carbon 290. Mass spectrometer 220 can be, for example, a Proline quadrupole vapor phase mass spectrometer available from Ametek Inc. of Berwyn, Pa. Mass spectrometer 220 as depicted in FIG. 2 can measure ethanol, water, and nitrogen concentrations.

At the top outlet of carbon bed 210 can be a valve cluster including valves V3, V9, and V4. As shown in FIG. 2, valve V3 can be in fluid communication with valve V10 as well as valve V12 which connects to heat exchanger 270. V12 and heat exchanger 270 can also act as a vent line. Valve V9 can be in fluid communication with a pressure indicator PI2 which is in electrical communication with data collector 230. Valve V4 can be in fluid communication with steam generator 240.

As shown in FIG. 2, at the bottom inlet of carbon bed 210 in addition to valves V16 and V1 can be a valve V2 in fluid communication with the inlet to heat exchanger 270. The outlet of heat exchanger 270 can feed a condensate collector 275. Between the outlet of heat exchanger 270 and condensate collector 275 can be a valve V13 in fluid communication with a particulate filter F2 and mass spectrometer 220 enabling measurement of the condenser outlet ethanol vapor concentration. Condensate collector 275 can be positioned on a scale 276 in electrical communication with data collector 230.

As shown in FIG. 2, apparatus 200 can further comprise a second heat exchanger 272, a first chiller 273, and a second chiller 274 all in fluid communication configured to supply heat exchanger 270 with cooling fluid.

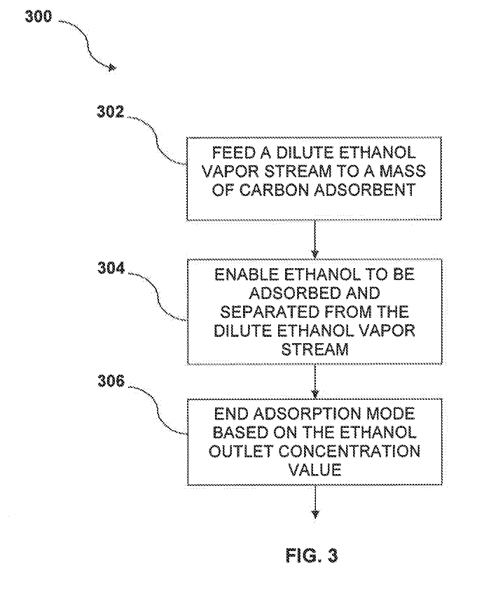

Apparatus 200 as described above can be configured to operate in both an adsorption mode 300 and a regeneration mode 400. Apparatus 200 can also be configured to operate in just adsorption mode 300 or regeneration mode 400 if desired. Adsorption mode 300, as shown in FIG. 3, can comprise steps 302, 304, and 306. Step 302 can comprise feeding a dilute ethanol air stream to a mass of carbon adsorbent. Step 304 can comprise enabling the ethanol to be adsorbed and separated from the air stream. Step 306 can comprise ending adsorption mode based on a minimum ethanol outlet concentration value (e.g., ethanol breakthrough). With regard to apparatus 200, adsorption mode step 302 can comprise, for example, of feeding nitrogen from nitrogen gas source 280 to ethanol sparger 250 and water sparger 260 producing an ethanol laden nitrogen stream which is supplied to carbon bed 210 containing a mass of carbon 290. Step 304 can comprise enabling within carbon bed 210 the ethanol and a portion of the water from the nitrogen steam to be absorbed by mass of carbon 290. Step 306 can be ended based on reaching a set point or threshold. For example, adsorption mode can be ended when a certain minimum concentration of ethanol (e.g., 200 ppm) is detected on the outlet of carbon bed 210 indicating breakthrough. Alternatively, adsorption mode can be ended when carbon bed 210 reaches ethanol saturation which is when the ethanol outlet concentration is equal to the ethanol inlet concentration indicating that no additional ethanol is being adsorbed by mass of carbon 290. Adsorption mode can continue beyond breakthrough and saturation however significant amounts of ethanol would be escaping carbon bed 210 resulting in low ethanol adsorption efficiency,

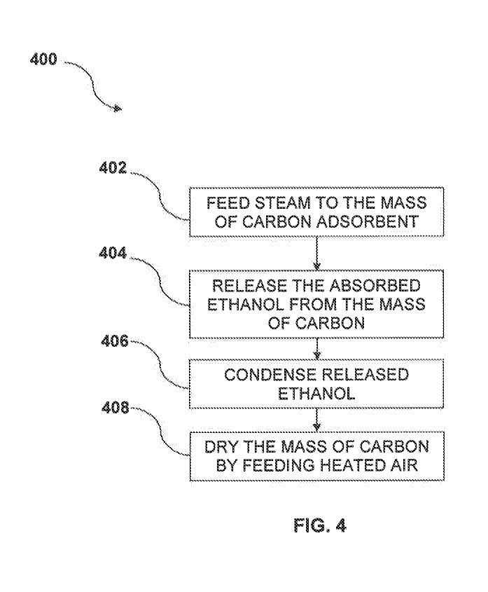

Regeneration mode 400 can be initiated after the conclusion of adsorption mode 300. Regeneration mode 400, as shown in FIG. 4, can comprise steps 402, 404, 406, and 408. Step 402 can comprise feeding steam to the mass of carbon adsorbent. Step 404 can comprise releasing the adsorbed ethanol from the mass of carbon adsorbent. Step 406 can comprise condensing the released ethanol using cooling water. Step 408 can comprise drying the mass of carbon adsorbent using heated air prior to the next adsorption cycle. With regard to apparatus 200, an exemplary embodiment, step 404, releasing the adsorbed ethanol can be accomplished by thermal regeneration. The thermal regeneration can comprise of generating steam using steam generator 240 and supplying that to the top of carbon bed 210 through valve V4. The line between steam generator 240 and valve V4 can be heated (e.g., to about 115° C.) and can include a condensate trap. Steam supplied to carbon bed 210 can heat mass of carbon 290 along with the adsorbed ethanol causing the ethanol to be desorbed and released from the mass of carbon 290. The released ethanol and steam is discharged as a vapor stream at the bottom of carbon bed 210 through valve V2 to heat exchanger 270. Heat exchanger 270 cools the vapor stream and condenses the ethanol and steam to form a condensate which is collected in condensate collector 275. Use of cooling water at 25° C. results in full condensation of ethanol (BP=78° C.) plus steam. The steam regeneration time can be determined on a mass of steam to a mass of carbon basis,

Following the thermal regeneration steps, regeneration mode 400 can further comprise drying mass of carbon 290 in order to remove residual moisture from within mass of carbon 290 and carbon bed 210. Drying can be accomplished in various ways. For example, drying can comprise introducing ambient air or gas (e.g., nitrogen) by way of valve V11, V8, and V1 into carbon bed 290. In another embodiment, drying can comprise supplying heated gas (e.g., nitrogen) by way of valve V16 into carbon bed 290. The line between nitrogen gas source 280 and valve V16 can be wrapped in heat tape to allow for heating of the nitrogen to an elevated temperature (e.g., about 75° C. 80° C.).

In another embodiment, pressure swing adsorption can be ullized rather than thermal adsorption/regeneration. Pressure swing adsorption can comprise of feeding the dilute ethanol vapor stream under high pressure to the absorbent media where it is attracted to the solid surfaces and becomes adsorbed. Once adsorbed the pressure can be reduced causing the release of the adsorbed gases. Pressure control as described above can be by way of compressors, pressurized gas sources, and valve control.

Following the conclusion of regeneration mode 400, carbon bed 210 can restart adsorption mode 300. This cycling between adsorption mode 300 and regeneration mode 400 can occur continuously. In another embodiment, apparatus 200 can comprise two carbon beds 210 and be configured such that the first carbon bed 210A can be operating in adsorption mode 300 while the second carbon bed 210B can be operating in regeneration mode 400 and then they can switch, enabling continuous feed of a solvent laden air stream to either the first carbon bed 210A or the second carbon bed 210B. Such configuration and operation can be advantageous from a production and efficiency standpoint because output capacity can be maximized as well as downtime minimized. In addition, both carbon beds 210 can utze the same steam generator 240, heat exchanger 270, and corresponding equipment.

As discussed above, carbon adsorbent can provide improved energy efficiency, particularly with regard to ethanol. Method 100 can, in an exemplary embodiment include a carbon adsorbent which exhibits high ethanol recovery efficiency, high ethanol adsorption capacity, and high ethanol selectivity (i.e., versus water adsorption).

Initially, apparatus 200 as described above was utilized to conduct adsorption mode 300 testing of method 100 on numerous carbon adsorbents to detect, record, and calculate the various performance characteristics including those listed above. The procedure and results of the testing is described below in greater detail.

### Experiment 1

Experiment 1 utilized apparatus 200 as described above to conduct adsorption mode 300 test on more than twelve carbon adsorbents to accurately detect and quantify the ethanol and water adsorption capacity, ethanol selectivity, and identify the initial ethanol breakthrough time and ethanol saturation time for these carbon adsorbents. The carbon adsorbents tested included two coconut shell, eight coal based, one wood based, and two polymer/resin.

For each carbon adsorbent tested, nitrogen was bubbled into ethanol sparger 250 and water sparger 260 at a controlled flow rate. The total nitrogen flow was based on a superficial velocity of 50 ft/min and the flow ratio of nitrogen into the ethanol sparger 250 and water sparger 260 was based on the desired ethanol inlet concentration into carbon bed 210. Ethanol sparger 250 and water sparger 260 were at a room temperature of 20 to 22° C. Carbon bed 210 was also at a room temperature of 20 to 22° C. while the top of carbon bed 210 was heated to greater than 22° C. to avoid vapor condensation. Mass spectrometer fluid communication lines were heated to about 110° C. to avoid vapor condensation.

Table 1 below lists the experimental parameters for Experiment 1, which remained constant for all the carbon adsorbent tests. The only parameter that changed was the carbon media tested and therefore the mass of carbon (Le., carbon loading) based on the given column dimensions. The carbon loading varied between 59-77 grams for the different carbons.

TABLE 1

| Parameter | Value |
|---|---|
| Superficial Velocity | 50 ft/min |
| Residence Time | 1.5 sec |
| Total Nitrogen (N2) Flowrate | 7.75 L/min |
| Ethanol Cylinder N2 Flowrate | 1.35 L/min |
| Water Cylinder N2 Flowrate | 6.40 L/min |
| Ethanol input concentration | 1.0 mol % (calculated) |
| Water input concentration | 1.98 mol % (calculated) |
| Nitrogen stream relative humidity (RH) | 82.5% (calculated) |
| Ethanol and water cylinder Temperature | 20-22 C. |
| Carbon Bed Temperature | 20-22 C. |
| Column Diameter | 1 inch |
| Column Length | 15 inches |
| Carbon Loading | 59-77 g |

Using the mass spectrometer data collected during each carbon test, the following values were either calculated or determined for each carbon adsorbent: ethanol adsorption capacity (g/g carbon), ethanol recovery efficiency (%), time to initial ethanol breakthrough (hr), and time to reach ethanol saturation (hr).

The ethanol adsorption capacity was calculated by subtracting the total ethanol inlet mass by the ethanol outlet mass. The ethanol inlet and outlet masses were calculated based on the area under the ethanol mass flow rate versus time profiles for carbon bed 210 inlet and outlet, based on mass spectrometry data.

The ethanol adsorption capacity was determined at initial ethanol breakthrough (i.e., initial time at which the ethanol outlet concentration is greater than mass spectrometer detection level 200 ppm) and ethanol saturation (i.e., time point at which ethanol outlet concentration is equal to ethanol inlet concentration). The water adsorption capacity was determined in a similar manner to ethanol initial breakthrough and ethanol saturation.

Table 2 below lists the top six of the more than fifteen carbon adsorbents tested and the time to breakthrough and saturation for each carbon, and the calculated adsorption capacity of ethanol and water for each carbon at breakthrough and saturation. As shown in Table 2, the carbon adsorbents tested included both coal (BX) and coconut shell (CS) carbons.

TABLE 2

| | | | | Carbon | | | Adsorption Capacity (g/g carbon) | | | |
|---|---|---|---|---|---|---|---|---|---|---|
| | Carbon Vendor | | | Loading | Residence Time (hr) | | Ethanol | | Water | |
| Log # | Name | Product | Type | (g) | Breakthrough | Saturation | Breakthrough | Saturation | Breakthrough | Saturation |
| 128-74 | Jacobi | Ecosorb | coal | 73.85 | 1.0 | 5.0 | 0.107 | 0.298 | 0.0182 | 0.1000 |
| 128-78 | Jacobi | Ecosorb | coconut | 73.66 | 2.2 | 3.2 | 0.231 | 0.297 | 0.0448 | 0.0650 |
| 128-80 | Carbtrol | | coconut | 73.50 | 1.7 | 3.1 | 0.181 | 0.267 | 0.0340 | 0.0566 |
| 128-81 | PES | | coal | 72.39 | 1.2 | 3.7 | 0.105 | 0.243 | 0.0299 | 0.0803 |
| 128-82 | Meadwestvaco | BX7540 | coal | 59.54 | 0.8 | 5.2 | 0.092 | 0.301 | 0.0253 | 0.1300 |
| 128-84 | Nichem | SLA-700 | coal | 77.27 | 1.5 | 4.5 | 0.142 | 0.284 | 0.0217 | 0.0670 |

As shown in Table 2, unexpectedly the Jacobi Ecosorb coconut shell (CS) carbon had the longest residence time before breakthrough at 2.2 hours with Carbtrol coconut shell (CS) carbon second at 1.7 hours and Nichem coal carbon third at 1.5 hours, However for residence time before saturation Meadwestvaco coal (BX) carbon exhibited the longest at 5.2 hours with Jacobi Ecosorb coal (BX) carbon second at 5 hours and Nichem coal (BX) carbon second at 4.5 hours.

With regard to the adsorption capacity of ethanol (g/g carbon) at breakthrough, Jacobi Ecosorb (CS) unexpectedly exhibited the highest ethanol adsorption capacity at breakthrough with a capacity of 0.231 g/g carbon. The second highest was the Carbtrol (CS) with 0.181 g/g carbon and third was Nichem (BX) at 0.142 g/g carbon. With regard to the adsorption capacity of ethanol (g/g carbon) at saturation, Meadwestvaco (BX) exhibited the highest ethanol adsorption capacity at saturation of 0.301 g/g carbon. The second highest was the Jacobi Ecosorb (BX) with 0.298 g/g carbon and third was the Jacobi Ecosorb (CS) at 0.297 g/g carbon.

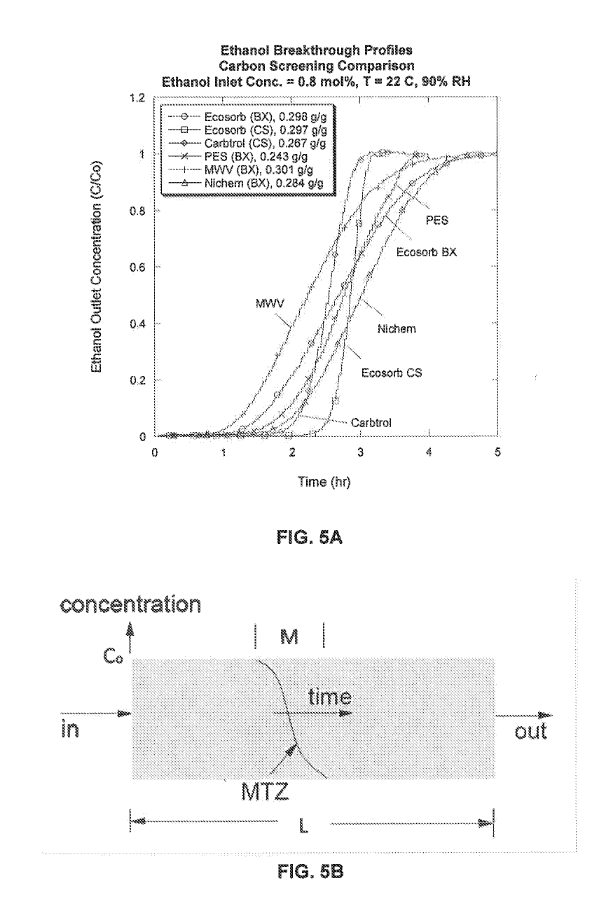

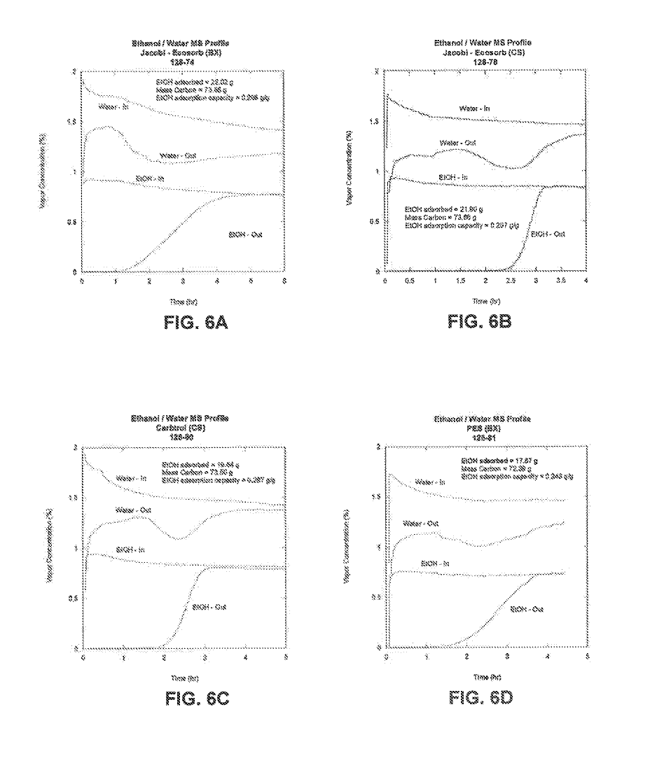

FIG. 5A shows ethanol breakthrough curves for the six carbon tests, showing ethanol outlet relative concentration (Outlet (C)/Inlet (Co)) versus time (hr). The breakthrough curves are based on a normalized outlet concentration, determined by dividing the outlet concentration (C) by the inlet concentration (Co). As shown in Table 2 and FIG. 5A, coconut shell (CS) type carbons (i.e., Ecosorb (CS) and Carbtrol (CS)) showed the longest time to initial breakthrough and a steep increase in ethanol outlet concentration until the ethanol saturation point was reached. Conversely, the coal based carbons exhibited a relatively short time to initial ethanol breakthrough (0.8-1.5 hrs) with a slower increase in ethanol outlet concentration until the ethanol saturation point. For the Ecosorb (CS), 78% of the ethanol adsorption saturation capacity was reached before initial ethanol breakthrough. For the Ecosorb (BX), 36% of the ethanol saturation capacity was reached before initial ethanol breakthrough. For the Carbtrol (CS), 68% of the ethanol saturation capacity was reached before initial ethanol breakthrough.

As discussed above, and unexpectedly, the coconut shell (CS) carbons exhibited a steeper increase in ethanol outlet concentration between breakthrough and saturation than that of the coal carbons (BX). To quantify this difference in performance exhibited between the different carbons a mass transfer zone (MTZ) length value was calculated for each carbon based on the data. Equation (1) and Equation (2) shown below were used to calculate the MTZ length. The variables used in calculating the MTZ length and their relationship are shown in FIG. 5B. Length (L) is the length of adsorbent bed, time (t) is time to reach concentration outlet (Co)/2 at bed outlet, and dt is the time from initial breakthrough until saturation.

$$\begin{matrix}MTZ Velocity ( u ) = \frac{Length ( L )}{Time ( t )} & Equation ( 1 ) \\ MTZ Length ( M ) = u \times dt & Equation ( 2 )\end{matrix}$$

Table 3 below shows the MTZ length in inches for the top six carbons. As shown numerically in Table 3 and visually in FIG. 5A, the MTZ length of Ecosorb (CS) is less than any of the other carbons and is substantially less than all the coal (BX) carbons. The lower the MTZ value at a given ethanol vapor feed concentration, the higher the ethanol breakthrough capacity, and the lower the steam regeneration energy per mass of ethanol desorbed (MJ/kg EtOH).

TABLE 3

| | | | | Ethanol Capacity | | |
|---|---|---|---|---|---|---|
| | | | | (g/g carbon) | | |
| | Carbon Vendor | | | Break- | Satu- | MTZ |
| Log # | Name | Product | Type | through | ration | (in) |
| 128-74 | Jacobi | Ecosorb | coal | 0.107 | 0.298 | 20.00 |
| 128-78 | Jacobi | Ecosorb | coconut | 0.231 | 0.297 | 5.56 |
| 128-80 | Carbtrol | | coconut | 0.181 | 0.267 | 8.75 |
| 128-81 | PES | | coal | 0.105 | 0.243 | 15.31 |
| 128-82 | Meadwestvaco | BX7540 | coal | 0.092 | 0.301 | 22.00 |
| 128-84 | Nichem | SLA-700 | coal | 0.142 | 0.284 | 15.00 |

Table 4 below, shows the ethanol selectivity as the ratio of ethanol to water adsorption selectivity at ethanol breakthrough and ethanol saturation, and the ethanol recovery efficiency at the initial ethanol breakthrough. It is believed that carbon with high ethanol selectivity results in a higher ethanol regeneration product concentration, a lower steam regeneration energy (MJ/kg EtOH), and a lower downstream purification energy requirement (i.e,, ethanol/water separation). As show in Table 4, these six carbons resulted in an ethanol recovery of greater than 99.3% up until initial ethanol breakthrough based on an ethanol detection Urnit of ˜200 ppm. The Nichem (BX) exhibited the highest ethanol to water adsorption ratio at a breakthrough of 6.54 with the Ecosorb (BX) coming in second at 5.88 and the Carbtrol (CS) in third at 5.32. The Ecosorb (CS) came in fourth at 5.16. However, due to the high MTZ value and low ethanol breakthrough capacity for Nichem (BX) and Ecosorb (BX), the stream regeneration energy (per mass of ethanol adsorbed) is expected to be significantly higher versus the Ecosorb (CS).

TABLE 4

| | Carbon Vendor | | | Ethanol/Water Adsorption Ratio | | Ethanol |
|---|---|---|---|---|---|---|
| Log # | Name | Product | Type | Breakthrough | Saturation | Recovery (%) |
| 128-74 | Jacobi | Ecosorb | coal | 5.88 | 2.98 | >99.5 |
| 128-78 | Jacobi | Ecosorb | coconut | 5.16 | 4.57 | >99.6 |
| 128-80 | Carbtrol | | coconut | 5.32 | 4.72 | >99.5 |
| 128-81 | PES | | coal | 3.51 | 3.03 | >99.5 |
| 128-82 | Meadwestvaco | BX7540 | coal | 3.64 | 2.32 | >99.4 |
| 128-84 | Nichem | SLA-700 | coal | 6.54 | 4.24 | >99.3 |

As part of the testing of each carbon, ethanol sparger 250 and water sparger 260 were weighed before and after each experiment in order to determine mass balance. Table 5 below shows the mass balance results for each trial comparing the liquid lost from the ethanol sparger 250 and water sparger 260 versus the ethanol and water inlet vapor mass totals measured by mass spectrometer 220.

TABLE 5

| | Carbon Vendor | | | Input | Sparger Mass (g) | | Mass Spectrometry (g) | | % Difference | |
|---|---|---|---|---|---|---|---|---|---|---|
| Log # | Name | Product | Type | Time (hr) | Ethanol | Water | Ethanol | Water | Ethanol | Water |
| 128-74 | Jacobi | Ecosorb | coal | 16.13 | 106.20 | 81.17 | 113.14 | 81.25 | −6.5 | −0.1 |
| 128-78 | Jacobi | Ecosorb | coconut | 14.50 | 98.26 | 75.89 | 108.50 | 74.18 | −10.4 | 2.3 |
| 128-80 | Carbtrol | | coconut | 19.00 | 121.65 | 92.16 | 133.40 | 91.01 | −9.7 | 1.2 |
| 128-81 | PES | | coal | 4.50 | 31.64 | 25.05 | 28.79 | 23.29 | 9.0 | 7.0 |
| 128-82 | Meadwestvaco | BX7540 | coal | 13.50 | 83.77 | 63.07 | 86.56 | 64.01 | −3.3 | −1.5 |
| 128-84 | Nichem | SLA-700 | coal | 4.70 | 34.92 | 25.52 | 35.24 | 26.74 | −0.9 | −4.8 |

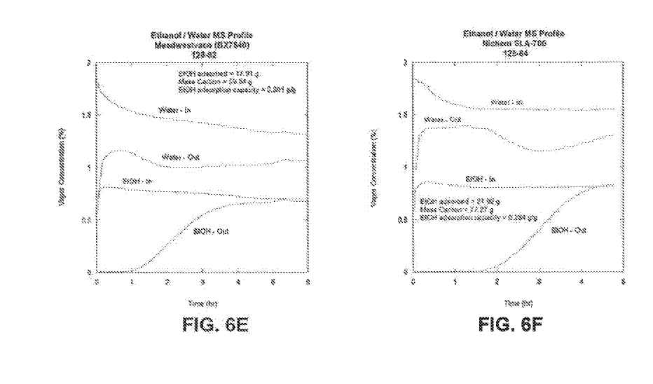

FIGS. 6A-6F show the ethanol and water mass spectrometer 220 concentration profiles for the inlet and outlet of carbon bed 210 for each test. As seen in the FIGS. 6A-6F the ethanol inlet profiles show a relatively constant concentration during the course of the tests. The ethanol outlet profiles initially show values less than the detection limit (˜200 ppm) of mass spectrometer 220, followed by initial ethanol breakthrough, then an “s-curve” increase in concentration to the ethanol saturation at which point the ethanol outlet concentration equals the ethanol inlet concentration. In FIGS. 6A-6F, the water inlet concentration profiles show an initial maximum value followed by a slow decrease while the water outlet shows an initial increase followed by a decrease in concentration corresponding to the ethanol breakthrough, as the mass of carbon approaches ethanol saturation and starts to adsorb more water. FIGS. 6A-6F help illustrate the benefit of ending the adsorption mode 300 at initial ethanol breakthrough minimizing the amount of water adsorbed to the carbon and maximizing ethanol regeneration product concentration.

All the different carbons tested were evaluated based on their adsorption capacity at breakthrough and saturation, residence time to breakthrough and saturation, ethanol to water adsorption ration, ethanol recovery efficiency, and the mass transfer zone length. Based on the recognized benefit of ending the adsorption mode 300 at initial ethanol breakthrough, the performance of the carbons at breakthrough became of particular interested. As a result, Ecosorb (CS) was selected for further testing given the fact it exhibited the highest ethanol adsorption capacity at breakthrough (0.231 g/g carbon), the longest residence time before breakthrough (2.2 hours), and the highest ethanol recovery efficiency (>99.6%). Although some of the other carbons exhibited higher ethanol to water adsorption ratio at ethanol breakthrough (e.g., Ecosorb (BX)=5.88, and Carbtrol (CS)=5.32, versus Ecosorb (CS)=5.16), it is believed that the lower ethanol breakthrough capacity and longer mass transfer zone length values of these carbons would result in higher ethanol regeneration energy and downstream energy requirements versus Ecosorb CS carbon (which showed the highest ethanol breakthrough capacity and shortest mass transfer zone).

Jacobi Ecosorb (CS) is available in varies particles sizes. For example, 3×6 mesh (3.35-6.30 mm), 4×8 mesh (2.36-4.75 mm), 6×12 mesh (1.70-3.35 mm), 8×16 mesh (1.18-2.36 mm), and other particles sizes, The Ecosorb (CS) utilized in Experiment 1 was the 4×8 mesh. Specifications for the Ecosorb (CS) include the following: CTC activity of min. 50%, Iodine number of min. 1000 mg/g, moisture content of max. 5%, total ash content of max. 4%, and ball-pan hardness of min 98%. Typical properties for the Ecosorb (CS) include surface area (BET) of 1100 m²/g, butane activity of 22%, and apparent density of 450 to 530 kg/m³.

In addition to utilizing a carbon adsorbent that provides improved efficiency as in Experiment I, it was also recognized energy efficiency may be improved by operating within specific VOC concentration ranges, specific temperature ranges, and utilizing certain steps as part of regeneration.

After testing numerous carbon adsorbents and selecting Ecosorb (CS), further testing was conducted on the Ecosorb (CS) in which the ethanol feed concentration was varied in order to evaluate its relationship to ethanol adsorption capacity and ethanol to water adsorption selectivity with the goal of further optimizing the downstream energy efficiency of method 100.

### Experiment 2

Experiment 2 utilized portions of apparatus 200, as described above, to perform adsorption mode 300 in order to evaluate the relationship between ethanol feed concentration and ethanol adsorption capacity and ethanol to water adsorption selectivity for Jacobi Ecosorb (CS).

Ecosorb (CS) was initially regenerated using a vacuum oven at 125° C., a vacuum pressure of 5 in-Hg, and a nitrogen purge of 10 liters per minute (LPM) for at least 2 hours to remove moisture content and impurities. After which, 65 g of Ecosorb (CS) was loaded into carbon bed 210. Similar to Experiment 1, nitrogen was bubbled into ethanol sparger 250 and water sparger 260 at controlled flow rates. The total nitrogen flow was based on a superficial velocity of 50 ft/min and the flow ratio of nitrogen into the ethanol sparger 250 and water sparger 260 was based on the desired ethanol inlet concentration into carbon bed 210. Table 6 below shows the nitrogen flow rates utilized for ethanol sparger 250 and water sparger 260 for each test. The ethanol feed concentration range was varied from 0.04 mol % to 1.8 mol % and feed relative humidity in nitrogen was varied from 98% to 83% based on the ethanol concentration range. The feed relative humidity was calculated based on the flow ratio of nitrogen in water sparger 260 divided by the total nitrogen flow.

As shown in Table 6, a total nitrogen flow rate of about 7.75 LPM was utilized representing a superficial velocity of 50 ft/min. For the adsorption tests done at 37° C. as shown in Table 6, the ethanol sparger 250 and water sparger 260 were heated to about 40 to 45° C. using heating plates under each sparger. For a couple of the tests the adsorption temperature was 22° C. as shown in Table 6. The tests done at 37° C. were to simulate mesophile conditions for the ethanol photobioreactor production process.

TABLE 6

| | Ethanol Feed | Nitrogen Flow (LPM) | | | RH |
|---|---|---|---|---|---|
| Lot # | (mol %) | Ethanol | Water | Total | (%) |

Adsorption Temperature = 37° C.

| | | | | | |
|---|---|---|---|---|---|
| 128-152 | 0.04 | 0.10 | 7.65 | 7.75 | 98.7 |
| 128-157 | 0.05 | 0.10 | 7.60 | 7.70 | 98.7 |
| 128-153 | 0.10 | 0.20 | 7.50 | 7.70 | 97.4 |
| 128-150 | 0.25 | 0.27 | 7.48 | 7.75 | 96.5 |
| 128-158 | 0.25 | 0.34 | 7.40 | 7.74 | 95.6 |
| 128-151 | 0.40 | 0.50 | 7.25 | 7.75 | 93.5 |
| 128-156 | 0.78 | 0.50 | 7.25 | 7.75 | 93.5 |
| 128-148 | 1.20 | 0.62 | 7.14 | 7.76 | 92.0 |
| 128-154 | 1.80 | 1.05 | 6.70 | 7.75 | 86.5 |

Adsorption Temperature = 22° C.

| | | | | | |
|---|---|---|---|---|---|
| 128-106 | 0.35 | 0.70 | 7.00 | 7.70 | 90.9 |
| 128-78 | 0.80 | 1.35 | 6.40 | 7.75 | 82.6 |

Note:
RH (%) = (Water N2 Flow/Total N2 Flow) * 100%

The vapor stream from ethanol sparger 250 and water sparger 260 were combined and fed into the bottom of carbon bed 210. Same as in Experiment 1, carbon bed 210 was oriented vertically to optimize carbon packing density. Heat jacket 211 was set to 40° C. and the temperature of mass of carbon 290 without adsorption was approximately 37.5° C. Heat of adsorption results in a temperature increase of about 2 to 8 C. The lines between ethanol sparger 250 and water sparger 260 and carbon bed 210, and vent hood 246 were heated using heat tape set to greater than 45° C. to avoid vapor condensation. As in Experiment 1, mass spectrometer 220 measured ethanol, water, and nitrogen concentrations. The lines feeding to mass spectrometer 220 were heated to about 110° C. to prevent vapor condensation.

Table 7 below lists the experimental parameters for Experiment 2. The ethanol input concentration and the nitrogen stream relative humidity varied, but the other parameters were maintained substantially constant. As shown in Table 6 and Table 7, the ethanol input concentration was increased from 0.04 mol % to 1.8 mol % (equivalent to an ethanol titer range of 0.148 to 6.7 wt % at 37° C.) for the testing.

TABLE 7

| Parameter | Value |
|---|---|
| Superficial Velocity | 50 ft/min |
| Residence Time | 1.5 sec |
| Total Nitrogen (N2) Flowrate | 7.75 L/min |
| Ethanol input concentration | 0.04-1.8 mol % |
| Nitrogen stream relative humidity (RH) | 82.5-98% (calculated) |
| Ethanol and water cylinder Temperature | 37 C. |
| Carbon Bed Temperature | 37 C. |
| Column Diameter | 1 inch |
| Column Length | 15 inches |
| Carbon Loading | 65 g |

Based on data collected during each test of Experiment 2, the ethanol to water selectivity and ethanol adsorption capacity were calculated for each test. The following nomenclature is used for the below equations: ethanol (EtOH), mass spectrometer (MS).

The ethanol saturation adsorpt on capacity (g/g carbon) was calculated by multiplying the total ethanol input to the carbon bed by the percent of ethanol input adsorbed divided by the carbon loading as represented by Equation 3 shown below.

$$EtOH Saturation Capacity = \frac{( \begin{matrix}Total EtOH Input ( Corr ) \times \\ \% EtOH Input Adsorbed\end{matrix} )}{Carbon Loading} \qquad Equation ( 3 )$$

The ethanol input to the carbon bed is corrected for the mass spectrometer 210 sample flow rate as shown below by Equation (4). The mass spectrometer 210 flow rate of about 0.4 LPM was about 5% of the total inlet vapor flow rate of 7.75 LPM.

Total EtOH Input(corr)=Total EtOH Input (g)×(100%−%MS Flowrate) Equation (4)

The percentage of ethanol input adsorbed on the carbon was determined from the mass spectrometer 210 ethanol inlet and outlet vapor profiles using Equation (5) shown below.

$$\% EtOH Adsorbed = [ \frac{( \begin{matrix}EtOH MS Inlet Area - \\ EtOH MS Outlet Area\end{matrix} )}{EtOH MS Inlet Area} ] \times 100 \% \qquad Equation ( 5 )$$

The carbon was weighed before and after adsorption to determine the total ethanol and water adsorbed at ethanol saturation. The water adsorption capacity was determined as the total ethanol and water adsorbed divided by the carbon loading, minus the ethanol saturation adsorption capacity as represented in Equation (6) below.

$$Water Capacity = ( \frac{EtOH + Water Adsorbed}{Carbon Loading} ) - EtOH Adsorption Capacity \qquad Equation ( 6 )$$

The percent ethanol adsorbed on the carbon at ethanol saturation was determined using Equation 7 shown below.

$$\% EtOH Adsorbed = ( \frac{EtOH Capacity}{( \begin{matrix}EtOH Capacity + \\ {H}_{2} O Capacity\end{matrix} )} ) \times 100 \% \qquad Equation ( 7 )$$

The ethanol breakthrough capacity was determined by multiplying the ethanol input mass flow rate (corrected) by the ethanol breakthrough time-point, divided by the carbon loading Equation (8) shown below.

$$EtOH Breakthrough Capacity = \frac{[ \begin{matrix}EtOH Input Mass Flow ( corr ) \times \\ t ( EtOH Breakthrough )\end{matrix} ]}{Carbon Loading ( g )} \qquad Equation ( 8 )$$

The water breakthrough capacity was determined using equations 9-11 below. The calculations assume a constant input of water and ethanol to the carbon bed based on a constant nitrogen flow to the water and ethanol cylinders, respectively. The % water adsorbed at ethanol breakthrough and the % water adsorbed at ethanol saturation are determined from the % difference in the inlet and outlet water mass spectrometer profiles at the ethanol breakthrough and ethanol saturation time points, respectively. For the equations below saturation=“Sat.” and breakthrough=“BT”.

$$\begin{matrix}Water Input @ EtOH Sat . ( g ) = \frac{( \begin{matrix}Water Sat . Cap . ( \frac{g}{g} Carbon ) \times \\ Carbon Loading ( g )\end{matrix} )}{( \frac{\% Water Adsorbed @ Sat .}{100 \%} )} & Equation ( 9 ) \\ Water Input @ EtOH BT = ( \frac{t ( EtOH BT}{t ( ETOH Sat )} ) \times Water Input @ Sa t . & Equation ( 10 ) \\ Water BT Capacity = \frac{( \begin{matrix}( Water Input @ EtOH BT \times \\ \% Water Adsorbed @ BT )\end{matrix} )}{Carbon Loading ( g )} & Equation ( 11 )\end{matrix}$$

Table 8 below shows the total ethanol and water adsorbed at ethanol saturation, the ethanol input to the carbon bed, the % ethanol adsorbed from the input (based on mass spectrometer data), and the % water adsorbed from input at ethanol breakthrough and ethanol saturation.

TABLE 8

| | | Total | | | | | |
|---|---|---|---|---|---|---|---|
| | Ethanol | EtOH + H20 | | | % Ethanol | % Water Adsorbed from | |
| Lot | Feed | Adsorbed | Ethanol Input to Bed (g) | | Adsorbed | Ethanol | Ethanol |
| # | (mol %) | (g)^{a} | Uncorrected^{b} | Corrected | from Input^{b} | Breakthrough | Saturation |

Adsorption Temperature = 37 C.

| | | | | | | | |
|---|---|---|---|---|---|---|---|
| 128-152 | 0.04 | 17.62 | 9.81 | 9.32 | 81.4 | 14.7 | 10.6 |
| 128-157 | 0.05 | 16.48 | 12.78 | 12.14 | 66.2 | 14 | 9.1 |
| 128-153 | 0.1 | 17.6 | 14.51 | 13.78 | 74.3 | 17.3 | 11.7 |
| 128-158 | 0.25 | 18.4 | 17.89 | 17.00 | 79.7 | 21.7 | 16.9 |
| 128-151 | 0.4 | 20.24 | 17.03 | 16.18 | 81.1 | 25.8 | 19.8 |
| 128-156 | 0.78 | 19.24 | 20.74 | 19.70 | 80.7 | 25 | 18.5 |
| 128-148 | 1.2 | 21.1 | 19.58 | 18.60 | 80.9 | 23.2 | 16.7 |
| 128-154 | 1.8 | 19.62 | 24.17 | 22.96 | 75.7 | 26.5 | 21.6 |

Adsorption Temperature = 22 C.

| | | | | | | | |
|---|---|---|---|---|---|---|---|
| 128-106b | 0.35 | 20.7 | 20.2 | 19.2 | 85.6 | 27.9 | 26 |
| 128-78c | 0.8 | 22.18 | 21.7 | 19.5 | 81.0 | 26.8 | 27.1 |

^{a}Total ethanol + water adsorbed on carbon at ethanol saturation.
^{b}At ethanol saturation

Table 9 shows the ethanol breakthrough and ethanol saturation time-points, the ethanol and water capacity at ethanol breakthrough and saturation, and the ethanol/water selectivity at ethanol breakthrough and saturation for experiments run at adsorption temperatures of 22° C. and 37° C.

TABLE 9

| | Ethanol | Adsorption Cycle Time (hr) | | Adsorption Capacity (g/g carbon) | | | | % Ethanol Adsorbed on | |
|---|---|---|---|---|---|---|---|---|---|
| Lot | Feed | Ethanol | Ethanol | Ethanol Breakthrough | | Ethanol Saturation | | Ethanol | Ethanol |
| # | (mol %) | Breakthrough | Saturation | Ethanol | Water | Ethanol | Water | Breakthrough | Saturation |

Adsorption Temperature = 37 C. ° ^{a}

| | | | | | | | | | |
|---|---|---|---|---|---|---|---|---|---|
| 128-152 | 0.04 | 7.00 | 11.8 | 0.085 | 0.127 | 0.117 | 0.154 | 40.1 | 43.0 |
| 128-157 | 0.05 | 7.00 | 14.8 | 0.088 | 0.096 | 0.124 | 0.130 | 47.8 | 48.8 |
| 128-153 | 0.10 | 5.50 | 10.0 | 0.117 | 0.092 | 0.158 | 0.113 | 55.9 | 58.2 |
| 128-158 | 0.25 | 3.60 | 6.3 | 0.149 | 0.055 | 0.208 | 0.075 | 73.2 | 73.6 |
| 128-151 | 0.40 | 2.60 | 3.9 | 0.166 | 0.095 | 0.202 | 0.110 | 63.6 | 64.8 |
| 128-156 | 0.78 | 1.70 | 2.8 | 0.182 | 0.042 | 0.245 | 0.051 | 81.4 | 82.7 |
| 128-148 | 1.20 | 1.35 | 1.9 | 0.203 | 0.092 | 0.232 | 0.093 | 68.9 | 71.3 |
| 128-154 | 1.80 | 0.90 | 1.5 | 0.212 | 0.025 | 0.267 | 0.034 | 89.3 | 88.6 |

Adsorption Temperature = 22 C.

| | | | | | | | | | |
|---|---|---|---|---|---|---|---|---|---|
| 128-106^{b} | 0.35 | 3.27 | 4.4 | 0.180 | 0.050 | 0.243 | 0.063 | 78.2 | 79.4 |
| 128-78^{c} | 0.80 | 2.20 | 3.2 | 0.148 | 0.059 | 0.215 | 0.086 | 71.5 | 71.3 |

^{a} Carbon Loadnig = 65.0 g (all experiments at adsorption temperature = 37 C. °)
^{b}Carbon Loading = 67.7 g
^{c}Carbon Loading = 73.66 g

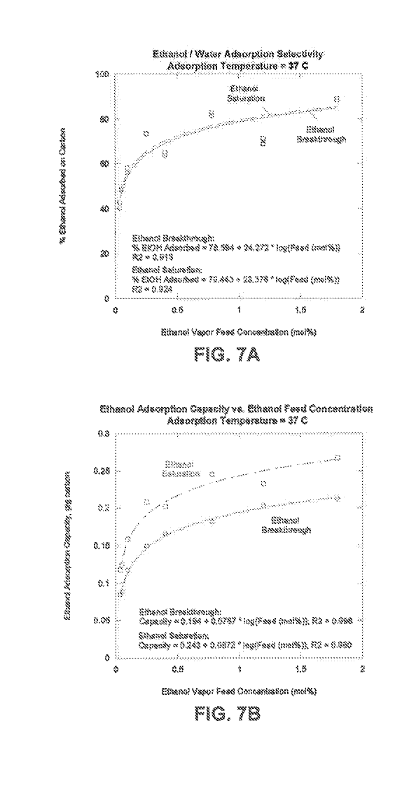

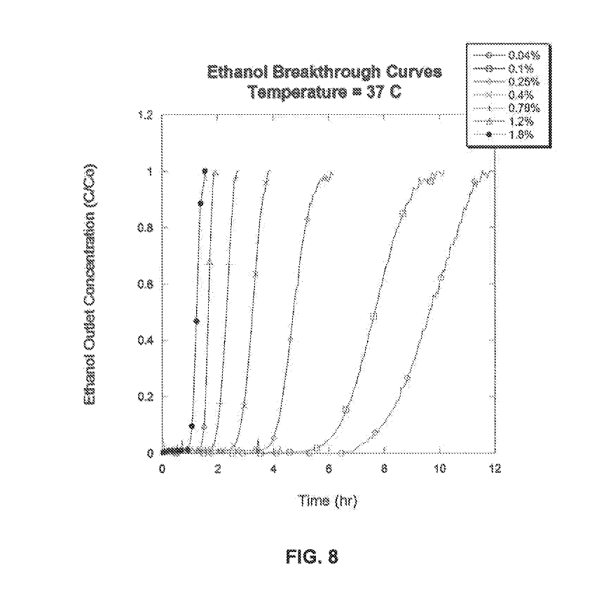

FIGS. 7A and 7B show plots of the ethanol I water adsorption selectivity and the ethanol adsorption capacity versus ethanol feed concentration over a range of 0.04-1.8 mol % (equivalent to concentration of 0.148 to 6.7 wt %) at an adsorption temperature of 37° C.

As shown in FIG. 7A, ethanol/water selectivity profiles at an adsorption temperature of 37° C. were essentially the same for ethanol breakthrough and ethanol saturation conditions. As shown in FIG. 7A, the percent ethanol adsorbed on carbon fits a logarithmic function of the ethanol feed concentration. The correlation for ethanol (EtOH) breakthrough and saturation are represented below by Equations 12 and 13.

EtOH Breakthrough: % EtOH Adsorbed=78.594+24.272×log EtOH Feed (mol %)) Equation (12)

EtOH Saturation :% EtOH Adsorbed=79.443+23.376×log EtOH Feed (mol %)) Equation (13)

As shown in FIG. 7B, as expected, the ethanol adsorption capacity was greater at ethanol saturation than ethanol breakthrough. The correlation for ethanol (EtOH) breakthrough and saturation are represented below by Equations 14 and 15.

$$\begin{matrix}EtOH Breakthrough \text{:} Ethanol Capacity ( \frac{g}{g} ) = 0.194 + 0.0787 \times log ( EtOH Feed ( mol \% ) ) & Equation ( 14 ) \\ EtOH Breakthrough \text{:} Ethanol Capacity ( \frac{g}{g} ) = 0.243 + 0.0872 \times log ( EtOH Feed ( mol \% ) ) & Equation ( 15 )\end{matrix}$$

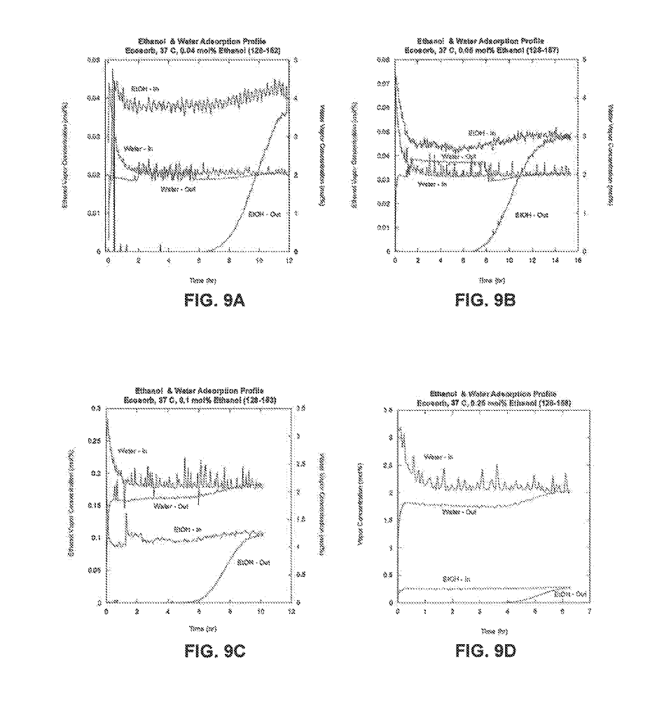

FIG. 8 shows plots of the ethanol breakthrough curves at the different concentrations for Experiment 2. As described above, the ethanol concentration ranged from 0.04-1.8 mol % at 37° C. As illustrated by FIG. 8, the higher the ethanol feed concentration the sooner ethanol breakthrough occurred.

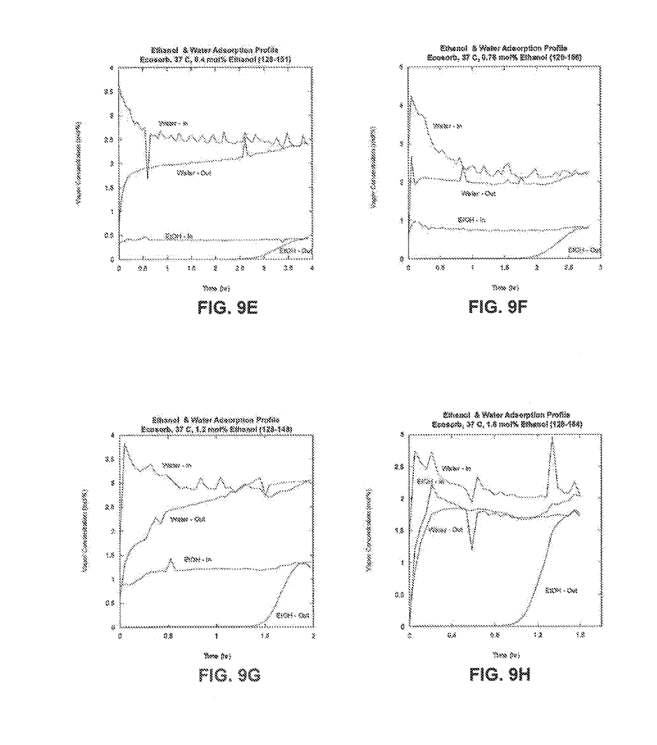

FIGS. 9A-9J show the ethanol and water concentration as measured by mass spectrometer for the different ethanol concentrations. FIGS. 9A-9H were for the tests at an adsorption temperature of 37° C. while FIGS. 9I and 9J were for the tests at an adsorption temperature of 22° C.

As described above, the results of Experiment 2 demonstrate that ethanol to water selectivity and ethanol adsorption capacity increased as a logarithmic function of the ethanol feed concentration at 37° C. More specifically, an increase in ethanol feed concentration from 0.04 mol % to 0.25 mol % resulted in an increase in % ethanol adsorbed from 40 to 73% at ethanol breakthrough, Furthermore, Experiment 2 demonstrated that ethanol feed concentrations of greater than 0.8 mol % resulted in greater than 80% ethanol adsorbed to carbon at ethanol breakthrough and ethanol saturation. As demonstrated by the results of Experiment 2, and surprisingly, the ethanol breakthrough capacity using Ecosorb (CS) was 70 to 80% of the ethanol saturation capacity over the ethanol feed concentration range tested. In addition, as shown in Table 9, ethanol to water selectivity and ethanol adsorption capacity were substantially equal at adsorption temperatures of 22° C. and 37° C. based on an ethanol inlet concentrate range of 0.35 to 0.8 mol %.

The results of Experiment 2 in which Ecosorb (CS) was tested, exhibit significant benefit by increasing the ethanol feed concentration and based on the performance benefit the downstream energy requirements.

### Experiment 3

Experiment 3 utilized apparatus 200 as described above to perform repeated cycles of adsorption mode 300 and regeneration mode 400 to evaluate the energy efficiency improvement based on the results of Experiments 1 and 2. For Experiment 3, Ecosorb (CS) was utilized and adsorption mode 300 was run with an ethanol vapor feed concentration of 0.54 mol % (equivalent to about 2 wt % ethanol titer) and an adsorption temperature of 22° C. Following adsorption mode 300, regeneration mode 400 was run as described above producing a condensate.

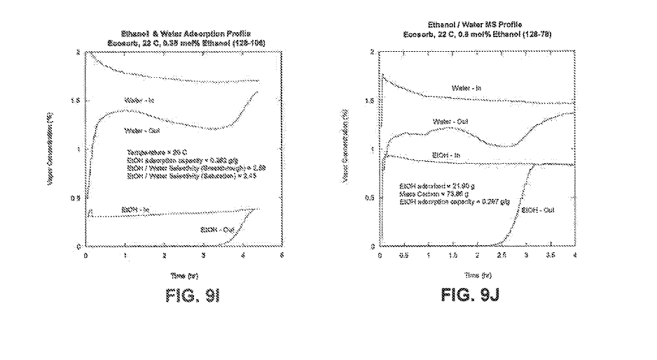

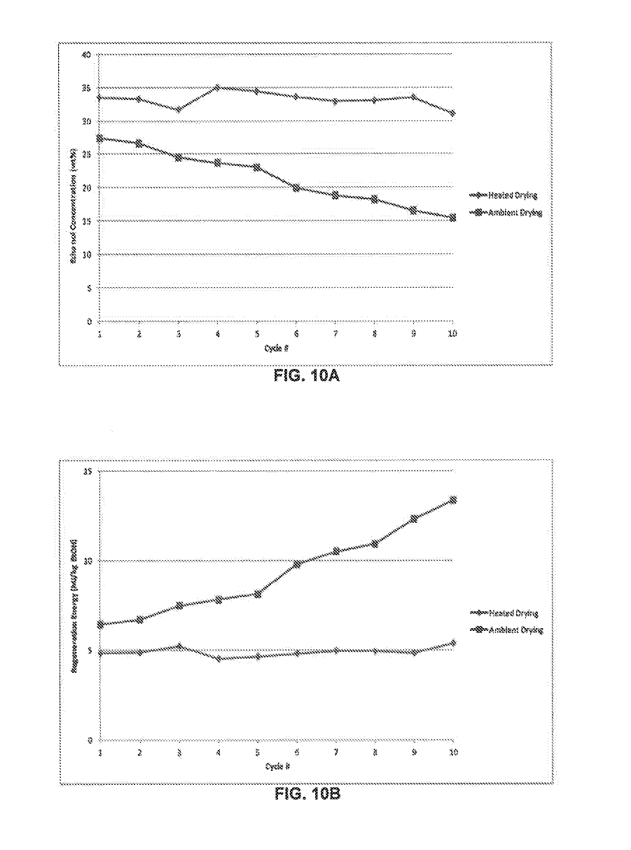

Phase 1 of Experiment 3 included 10 cycles with heated air drying and 10 cycles with ambient air drying. For the ambient air runs the steam regeneration time was 5 minutes and for the heated air runs the steam regeneration time was 25 minutes. FIG. 10A plots the ethanol condensate concentration following regeneration for the 10 cycles of ambient air drying and 10 cycles of heated air drying. As shown in FIG. 10A, for the heated drying the condensate ethanol concentration maintained a value between 30 and 35 wt % for all 10 cycles whereas the ambient drying initially exhibited a condensate ethanol concentration of about 27 wt %, but that steadily dropped to 15 wt % by the tenth cycle. Accordingly, the heated air drying and longer steam regeneration produces consistently higher condensate ethanol concentration.

FIG. 10B is a plot of steam regeneration energy (MJ/kg EtOH) for the 10 cycles of ambient drying and 10 cycles of heated drying. As shown in FIG. 10B, for the heated drying the steam regeneration energy maintained a value of about 5 MJ/kg EtOH for the 10 cycles whereas the ambient drying initially exhibited a value of about 7 MJ/kg EtOH, but that steadily increased to more than 12 MJ/kg EtOH by the tenth cycle. The increase in the steam regeneration energy for the ambient air drying can be attributed to the accumulation of water on the carbon (i.e., 0.45 g waterig carbon after 10 cycles) and a decrease in ethanol working capacity (0.102 to 0.062 g/g carbon).

Phase 1 of Experiment 3 illustrates the significant benefit of heated air drying both on the condensate ethanol concentration as well as the regeneration energy. The results of Experiment 3 illustrated in FIGS. 10A and 10B are based on cycling experiments using an adsorption temperature of 22° C. The regeneration comprised a steam loading of 0.30 kg steam per kg carbon and resulted in an ethanol working capacity of 0.16 kg/kg carbon. As shown in FIGS. 10A and 10B, this translated to a steam energy requirement of about 5 MJ/kg EtOH and a condensate ethanol concentration of 33 wt %, which was more than 15X concentration of the photobioreactor titer of 2 wt %.

Phase 2 of Experiment 3 testing included performing an adsorption and regeneration mode wherein the adsorption temperature was 37° C., the regeneration comprised a steam loading of 0.17 kg steam per kg carbon and resulted in an ethanol working capacity of 0.08 kg/kg. These results translated to a steam energy requirement of 5.1 MJ/kg EtOH and an ethanol condensate concentration of 32 wt %, representing a concentration factor more than 15X from the 2 wt % ethanol titer. The concentration factor of the ethanol condensate versus the ethanol feed concentration can vary. For example, the concentration factor can be about 10X, 12X, 14X, 15X, 16X, 18X, 20X. Equations 16 to 20 were used to calculate the steam energy requirement for a given ethanol titer and vapor phase concentration. Equation 17 is based on an ethanol/water/air vapor liquid equilibrium model (Aspen Plus) at a temperature of 37° C. For example, based on an ethanol titer of 2 wt % (i.e., corresponding to an ethanol vapor phase concentration of 0.54 mol % at vapor liquid equilibrium), EtOH working capacity of 0.08 g/g carbon, steam loading of 0.17 kg/kg carbon, steam enthalpy value of 2.085 MJ/kg steam, and natural gas efficiency of 85.7% the resulting steam energy requirement is 5.1 MJ/kg EtOH as shown below.

$$\begin{matrix}EtOH Saturation Capacity = 0.245 + ( 0.0928 \times log ( EtOH Vapor ( mol \% ) ) & Equation ( 16 ) \\ EtOH Vapor ( mol \% ) = 0.269 \times EtOH titer ( wt \% ) & Equation ( 17 ) \\ EtOH Working Capacity = 0.36 \times Saturation Capacity & Equation ( 18 ) \\ EtOH Regeneration Conc . = ( \frac{Working Capacity}{( EtOH Working Cap . \times Steam Loading )} ) \times 100 \% & Equation ( 19 ) \\ Steam Energy ( \frac{MJ}{kg} EtOH ) = \frac{Steam Loading \times Steam Enthalpy}{EtOH Working Cap . \times Natural Gas Eff .} = 5.1 \frac{MJ}{kg} EtOH & Equation ( 20 )\end{matrix}$$

The unexpectedly low steam loading value of 0.17 kg/kg carbon to meet an ethanol working capacity of 0.08 kg/kg carbon resulted in a reduction of steam energy by about 50% when compared to a value of 10.6 MJ/kg EtOH, which was the initial estimate based on vendor design recommendations of 0.32 kg/kg for a steam loading value and an ethanol working capacity of 33% of the ethanol saturation capacity.

As described above, distillation energy can constitute a significant portion of the enthalpy of EtOH when distillation is used to concentrate a dilute ethanol stream (e.g., 0.2 wt % to 6.7 wt %) to a fuel grade concentration (e.g., greater than 98.7 wt %). But by utilizing apparatus 200 and method 100 as described above, the dilute ethanol stream can first be concentrated by more than 15X before distillation, drastically reducing the energy required by distillation.

### Experiment 4-Pilot Scale

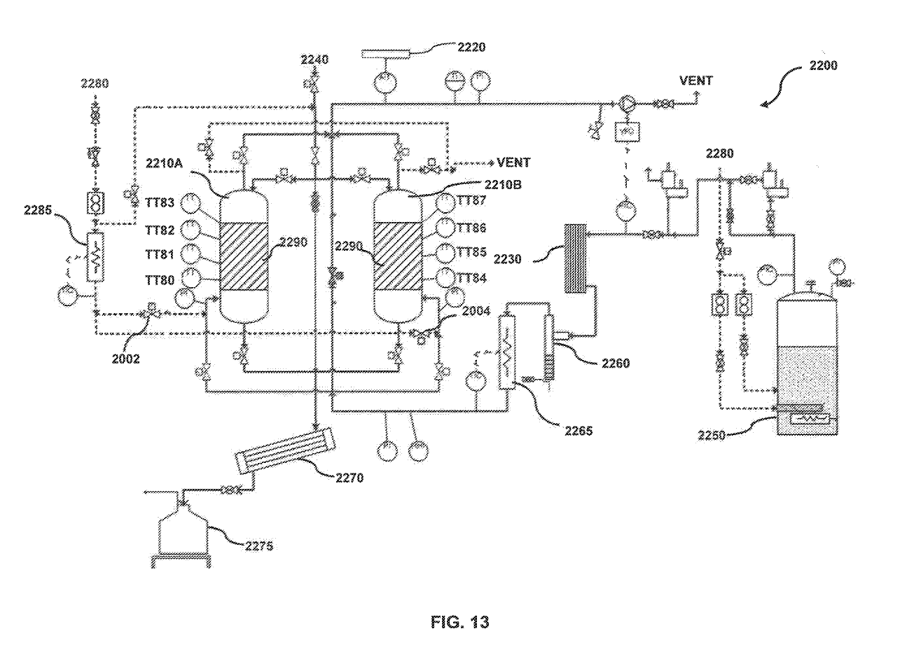

To evaluate and assess the scale up viability of adsorption mode 300 and regeneration mode 400, a pilot scale regeneration apparatus 2200 similar to apparatus 200 was assembled, an exemplary flow diagram of which is shown in FIG. 13. For Experiment 4, apparatus 2200 had a carbon loading of about 15.5 kg per carbon bed while apparatus 200 had a carbon loading of 0.395 kg per carbon bed, thereby apparatus 2200 had a carbon loading of about 39X that of apparatus 200.

As shown in FIG. 13, apparatus 2200 may comprise a first carbon bed 2210A and a second carbon bed 2210B, a Flame Ionization Detector (FID) 2220, a steam source 2240, an ethanol in water bubbler 2250, a heat exchanger 2270, and a gas source 2280.

As shown in FIG. 13, apparatus 2200 can be assembled such that gas source 2280 can be in fluid communication with the inlet of ethanol and water bubbler 2250 as well as the bottom of carbon beds 2210A and B through valve 2002 and valve 2004. Gas supplied to ethanol and water bubbler 2250 from gas source 2280 can be configured to generate a dilute ethanol laden vapor stream. The outlet of ethanol in water bubbler 2250 can be in fluid communication with the bottom of carbon beds 2210A and B via a condenser 2230, a gas liquid separator 2260 a conditioning heater 2265, and interconnecting piping. In addition, apparatus 2200 may include a plurality of valves (e.g., isolation valves, pressure relief valves, sampling valves, etc.) and a plurality of instruments (e.g., pressure indicating controllers, temperature indicating controllers, pressure indicators, etc.). It is contemplated that the configuration of valves, instruments, and other components of the apparatus may vary. For example, in some embodiments, condenser 2230 and/or separator 2260 may be removed.

Gas from gas source 2280 can be bubbled into ethanol water bubbler 2250 at a controlled flow rater using a flow controller. The dilute ethanol vapor stream produced by ethanol in water bubbler 2250 can be supplied to carbon bed 2210A and/or carbon bed 2210B. Carbon Beds 2210A and B can each be 8 inches in diameter by 36 inches in length and configured to receive a mass of carbon 2290. For Experiment 4 the mass of carbon 2290 was Jacobi Ecosorb (CS).

Carbon beds 2210A and B can each comprise temperature transmitters, for example carbon bed 2210A can include temperature transmitters TT80, TT81, TT82, and TT83 and carbon bed 2210B can include temperature transmitters TT84, TT85, TT86, and TT87. The temperature transmifters can read the temperature within each carbon bed at the inlet, outlet, and within each bed.

As shown in FIG. 13, FID 2220 may be in fluid communication with carbon beds 2210A and B and configured to detect ethanol breakthrough during adsorption mode 300. The bottom of each carbon bed 2210A and B can be in fluid communication with heat exchanger 2270 and condensate collector 2275, thereby enabling condensing and capture of the desorbed ethanol vapor stream during steam regeneration mode 400.

Apparatus 2200 can be configured to operate in adsorption mode 300 and regeneration mode 400, as described herein. For apparatus 2200, step 302 of adsorption mode can comprise of feeding the dilute ethanol vapor stream to the mass of carbon 2290 in either carbon bed 2210A or 2210B. Step 304 can comprise of enabling the ethanol to be adsorbed by the mass of carbon 2290 from the vapor stream. Step 306 can comprise of ending adsorption mode based on a minimum ethanol outlet concentration value (e.g., ethanol breakthrough) as detected by FID 2220. Alternatively, adsorption mode 300 may be ended when carbon bed 2210A or 2210B reaches ethanol saturation. Adsorption mode 300 may continue beyond breakthrough and saturation, however significant amounts of ethanol would be escaping carbon bed 2210A or 2210B resulting in low ethanol adsorption efficiency.

Regeneration mode 400 can be initiated after the conclusion of adsorption mode 300. For apparatus 2200, step 402 of regeneration mode 400 can comprise of feeding steam from steam source 2240 to the mass of carbon 2290. Step 404 can comprise releasing the adsorbed ethanol from the mass of carbon 2290. Step 406 can comprise condensing the released ethanol using heat exchanger 2270. Step 408 can comprise drying mass of carbon 2290 prior to the next adsorption cycle, using for example, gas from gas source 2280. The gas may be heated prior to being supplied to carbon bed 2210A or 2210B, for example using an inline drying heater 2285.

Following the conclusion of regeneration mode 400, carbon bed 2210A and/or B can restart adsorption mode 300. This cycling between adsorption mode 300 and regeneration mode 400 can occur continuously. As shown in FIG. 13, apparatus 2200 includes two carbon beds 2210A and B, thereby enabling first carbon bed 2210A to operate in adsorption mode 300 while the second carbon bed 2210B can operate in regeneration mode 400 and then they can switch, enabling continuous feed of a solvent-laden air stream to either the first carbon bed 2210A or the second carbon bed 2210B,

For experiment 4, pilot scale apparatus 2200, was operated in adsorption mode 300 and regeneration mode 400 with carbon bed 2210B online using Jacobi Ecosorb (CS), The testing parameters and results for Experiment 4 are shown below in Table 10 along with the corresponding results for the Jacobi Ecosorb (CS) from Experiment 3 utilizing lab scale apparatus 200.

TABLE 10

| | | Apparatus | Apparatus |
|---|---|---|---|
| Parameter | Units | 200 | 2200 |

Column Characteristics

| | | | |
|---|---|---|---|
| Column Diameter | inches | 1.5 | 8 |
| Column Bed Depth | inches | 36 | 36 |
| Carbon Loading | kg | 0.395 | 15.5 |

Adsorption Mode 300

| | | | |
|---|---|---|---|
| Ethanol Titer | wt % | 2 | 1.32 |
| Air Row Rate | LPM | 23 | 1400 |
| Ethanol Vapor Feed Concentration | mol % | 0.54 | 0.36 |
| Feed Relative Humidity | RH % | 90 | 50 |
| Adsorption Temperature | C | 37 | 37 |
| Ethanol Breakthrough Capacity | g/g carbon | 0.2 | 0.18 |

Regeneration Mode 400

| | | | |
|---|---|---|---|
| Steam Loading | g/g carbon | 0.17 | 0.17 |
| Ethanol Working Capacity | g/g carbon | 0.082 | 0.07 |
| Ethanol Condensate Concentration | wt % | 32 | 29 |
| Regeneration Steam Energy | MJ/kg EtOH | 5.1 | 5.8 |
| Ethanol Concentration Factor | | 16 | 22 |

As indicated in TABLE 10, carbon loading for Experiment 4 was 15.5 kg, the ethanol titer was 1.32 wt %, the air flow rate was 1400 LPM producing an ethanol vapor feed concentration of 0.36 mol %. The ethanol breakthrough capacity for Experiment 4 was 0.18 g/g carbon. The 10% lower ethanol breakthrough capacity for the Jacobi Ecosorb (CS) for Experiment 4 than observed in Experiment 3 was expected based on the lower ethanol vapor feed concentration.

For regeneration mode 400, a steam loading of 0.17 g steamig carbon was ufilized for Experiment 4. This resulted in an ethanol condensate concentration of 29 wt % using apparatus 2200 versus 32 wt % for apparatus 200 with an equivalent steam loading of 0.17 g steamig carbon. This translated to an ethanol concentration factor of 22X for apparatus 2200 versus 16 for apparatus 200. The associated steam regeneration energy was 5.8 MJ/kg EtOH for apparatus 2200 versus 5.1 MJ/kg EtOH for apparatus 200. Therefore, although the steam regeneration energy was higher for apparatus 2200, due to the lower ethanol vapor feed concentration, the ethanol concentration factor was also higher.

In summary, Experiment 4 demonstrated that pilot scale apparatus 2200 performed comparable to lab scale apparatus 200 in terms of ethanol product concentration and regeneration steam energy, thereby demonstrating the scale up viability of adsorption mode 300 and regeneration mode 400 utilizing the Jacobi Ecosorb (CS). It is contemplated that the pilot scale apparatus 2200 and method for operating disclosed herein may be further scaled up to increase production capacity of the ethanol condensate. For example, the carbon loading may be increased 50X, 100X, 200X, 500X.

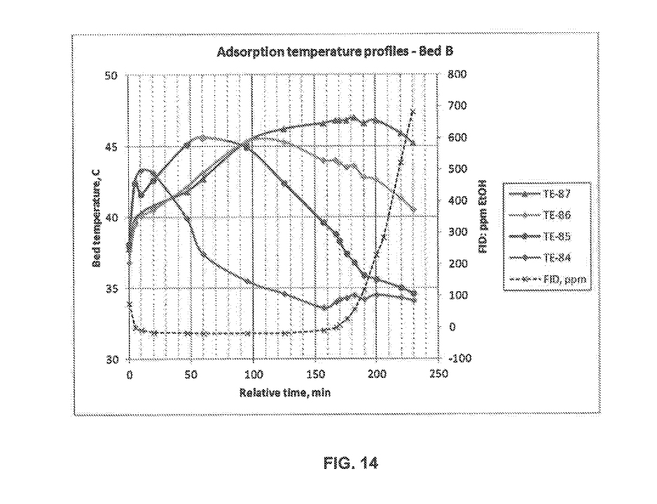

FIG. 14 is a plot of the ethanol breakthrough curve on the right axis and the ethanol adsorption temperature profiles of carbon bed 2210B on the left axis for Experiment 4. As shown in the plot, ethanol breakthrough occurred at about 170 minutes coinciding with a maximum temperature at the top of the carbon bed (Le. TE-87).

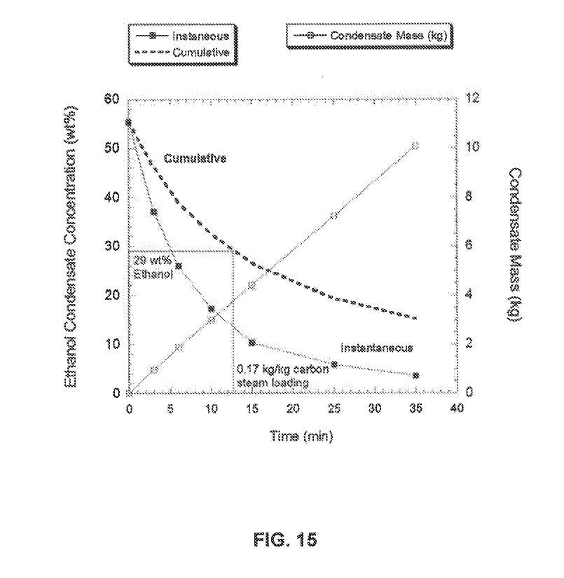

FIG. 15 is a plot of the ethanol steam regeneration results showing the instantaneous (measured) and the cumulative (calculated) ethanol condensate concentration (wt %) on the left axis, and the condensate mass (i.e., ethanol and water) on the right axis. As shown in FIG. 15, the ethanol is shown to initially desorb at a high concentration, then follow an exponential decrease as the steam regeneration continues. The resulting cumulative ethanol condensate concentration was 29 wt % at a steam loading of 0.17 g steam/g carbon.

### Falling Microbeads

According to another exemplary embodiment, a falling microbead counter-flow process and system was employed to improve the energy efficiency of method 100, with respect to ethanol vapor recovery.

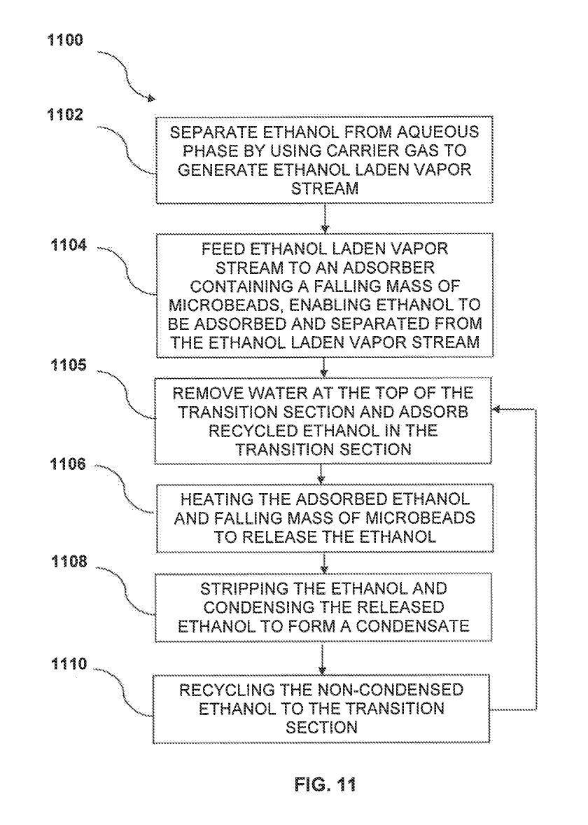

According to an exemplary embodiment a method 1100 of recovering and concentrating ethanol from a dilute ethanol aqueous phase is depicted as a flow chart in FIG. 11 and described below in more detail. Method 1100 can comprise the steps of 1102, 1104, 1105,1106, 1108, and 1110. Step 1102 can comprise separating ethanol from the aqueous phase by using a carrier gas to generate an ethanol laden vapor stream. Step 1104 can comprise feeding the ethanol laden vapor stream to an adsorber containing a falling mass of microbeads enabling the ethanol to be absorbed and separated from the ethanol laden vapor stream. Step 1105 can comprise removing desorbed water at the top of a transition section using a recycled inert purge gas stream and adsorbing recycled ethanol in the transition section. Step 1106 can comprise heating the adsorbed ethanol and the falling mass of microbeads to release the ethanol. Step 1108 can comprise stripping the released ethanol using an inert gas (Le. CO₂ or N₂) and condensing the released ethanol to form a condensate. Step 1110 can comprise of recycling the non-condensed ethanol to the bottom of the transition section. In another embodiment, method 1100 can comprise of receiving an ethanol laden vapor stream rather than separating ethanol from the aqueous phase by using a carrier gas.

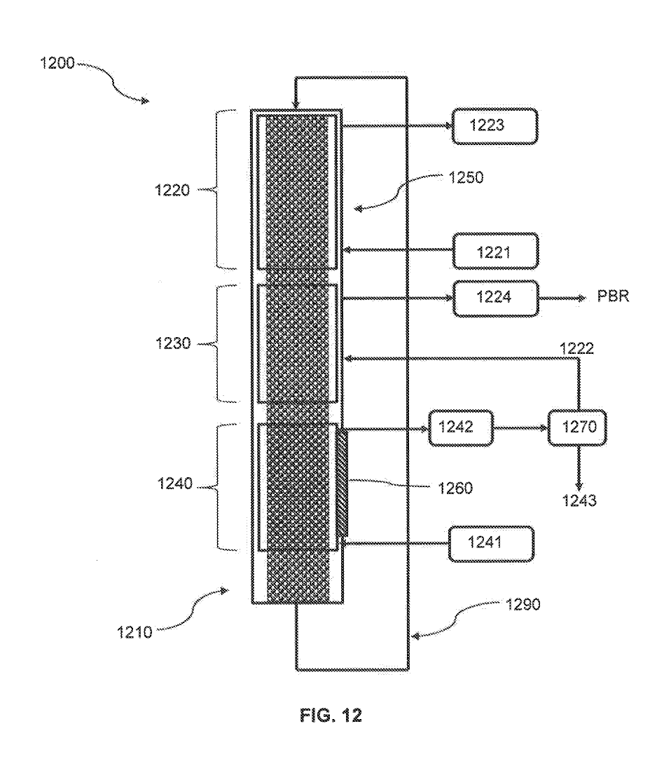

According to an exemplary embodiment, a system 1200 as shown in FIG. 12 can be configured to perform method 1100, as described above. System 1200 can comprise a column 1210 containing at least an adsorber 1220, a transition 1230, and a stripper 1240 all of which can be in fluid communication. As shown in FIG. 12, adsorber 1220 can be positioned above transition 1230, and transition 1230 can be above stripper 1240.

System 1200 can further comprise a dilute ethanol vapor stream 1221 in fluid communication with adsorber 1220. As shown in FIG. 12, dilute ethanol vapor stream 1221 can be supplied to the lower region of adsorber 1220.

System 1200 can further comprise a plurality of microbeads 1250 configured to fall through column 1210 and adsorb and desorb the ethanol from dilute ethanol vapor stream 1221. Adsorber 1220 is configured to receive dilute ethanol vapor stream 1221 and direct it up vertically through the adsorber while a plurality of microbeads 1250 fall down through adsorber 1220. In the presence of this counter-flow interaction, the ethanol can be adsorbed by the plurality of microbeads 1250 and a depleted dilute aqueous ethanol vapor stream 1223 can be vented at the upper region of adsorber 1220.

Microbeads 1250 can be hard and resilient allowing for repeated cycling through system 1200 without degradation. Microbeads 1250 can be configured for fast adsorption and desorption. In addition, microbeads 1250 can have a low heat of adsorption.

Adsorber 1220 can contain an internal packing structure configured to enhance the ethanol adsorption by microbeads 1250 distribution and ethanol vapor adsorption efficiency. The internal packing structure can promote uniform flow of falling microbeads 1250 while minimizing pressure drop. For example, pressure drop can be less than about 0.04 psi, 0.05 psi, 0.06 psi, 0.07 psi, 0.08 psi, 0.09 psi, or 0.1 psi. The minimal pressure drop can translate to a reduction in energy consumption (e.g., blower energy).

System 1200 can further comprise an inert stripper gas stream 1241 (e.g., N₂ or CO₂) in fluid communication with stripper 1240. As shown in FIG. 12, stripper gas stream 1241 can be configured to supply a stripper gas to the lower region of stripper 1240. An inert stripper gas can be used to mitigate potential ethanol flammability concerns.

System 1200 can further comprise a heat source 1260 configured to heat at stripper 1240. Heat source 1260 can be configured to heat stripper 1240 and also heat microbeads 1250 and the adsorbed ethanol as they fall through stripper 1240. By heating microbeads 1250, the ethanol adsorbed can be desorbed and thus released. Stripper gas stream 1241 supplied to stripper 1240 can flow vertically upward and collect the desorbed ethanol and be discharged as stream 1242 from he upper region of stripper 1240, as shown in FIG. 12.

Heat source 1260 can be configured for indirect heating, such that heat source 1260 does not directly contact microbeads 1250, stripper gas stream 1241, and the ethanol. For example, heat source 1260 can comprise heat trace wrapped around the stripper, steam circulated around the stripper, or the stripper could consist of a tube and shell heat exchanger where steam is supplied to an outer shell while microbeads 1250, inert stripper gas stream 1241, and the ethanol are all contained within the inner tube. Use of indirect heating can result in a maximum ethanol production concentration based on the high ethanol to water adsorption selectivity.

The flow of microbeads 1250 in the stripper section can be characterized as a moving bed, which can provide the required residence time for efficient ethanol desorption.

Transition 1230 can utilize the stratified temperature profile to efficiently remove water from the microbeads at the top of 1230 since the stripping temperature for water is less than that of ethanol. This can enable the separation of at least a portion of the water vapor prior to desorption and collection of the ethanol, resulting in an enhanced ethanol production concentration above the ethanol to water adsorption selectivity ratio. The recycled non-condensed ethanol 1222 can be adsorbed in the transition section 1230.

System 1200 can further comprise a condenser 1270 configured to receive the ethanol from stream 1242 discharged from stripper 1240. Condenser 1270 can cool the ethanol and form a condensate 1243. Non-condensed ethanol 1222 can be recycled to the bottom of the transition section 1230.

System 1200 can further comprise a transport apparatus 1290 configured to transport microbeads 1250 from the bottom of column 1210 back to the top of column 1210. Transport apparatus 1290 can be configured for continuous operation and enable continuous operation of system 1200. For example, transport apparatus 1290 can comprise a pneumatic air lift.

System 1200 as described above can be configured to receive dilute ethanol vapor 1221 from an ethanol photobioreactor production system. System 1200 can be configured such that stripper gas stream 1241 can be CO₂ and CO₂ 1224 can be recycled back to the ethanol photobioreactor (PBR) production system. Stream 1224 can provide photobioreactor make-up water from desorption in the transition section. System 1200 can also be configured such that the depleted ethanol vapor stream 1223 discharged from the top of adsorber 1220 can be recycle back upstream to the ethanol photobioreactor production system.

According to various embodiments, the concentration of ethanol titer from the ethanol photobioreactor production system can be about 0.15 wt % to about 6.7 wt %. System 1200 can be configured such that based on an ethanol vapor feed concentration 1221 of between 0.04 mol % to 1.8 mol %, condensate 1243 can have an ethanol concentration in the range of, for example, 80 wt % to 95 wt %, or 85 wt % to 95 wt %, or 90 wt % to 95 wt %. Achieving such a high ethanol condensate concentration can be the elimination of the traditional distillation step, which consumes significant energy. In addition, due to the stripper product stream temperatures of system 1200 at 150° C., system 1200 can be integrated with a molecular sieve without significant or potentially any intermediate heat treatment between system 1200, and the molecular sieve. The molecular sieve can be configured to increase the ethanol concentration to achieve fuel grade (e.g., greater than 98.5%).

In other embodiments, system 1200 as described herein can be configured such that preconditioning steps required for static bed adsorption processing with high relative humidity feed streams can be eliminated resulting in further decrease in ethanol energy recovery requirements, due to water removal in the transition section and further water removal in the stripper section.

Now referring back to method 1100 shown in FIG. 11, method 1100 can be executed such that each step is performed simultaneously and continuously by different portions of system 1200.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

1.-41. (canceled)

42. A method for recovering a volatile organic compound (VOC) from a VOC laden vapor stream comprising:
feeding the VOC laden vapor stream to an adsorber containing a falling mass of microbeads, enabling the VOC to be absorbed and separated from the VOC laden vapor stream;
heating the adsorbed VOC and the falling mass of microbeads to release the VOC; and
stripping and condensing the released VOC to form a condensate.

43. The method of claim 42, wherein the VOC and the falling mass of microbeads are heated by indirect contact using steam.

44. The method of claim 42, wherein each step is performed simultaneously and continuously.

45. The method of claim 42, wherein the VOC is ethanol and the concentration in the vapor stream is about 0.01 mol % to about 0.8 mol %.

46. The method of claim 42, wherein the VOC is ethanol and the ethanol vapor stream is a product of a photobioreactor process.

47. The method of claim 42, further comprising removing the adsorbed water from the falling mass of microbeads to release and separate at least a portion of the water before releasing the adsorbed VOC.

48. The method of claim 42, wherein stripping comprises feeding an inert stripper gas stream counter-flow to the falling mass of microbeads to capture the released VOC and supply it to a condenser; wherein the VOC is ethanol and the ethanol vapor stream is a product of a photobioreactor process, and the inert stripper gas stream used for stripping is CO2 that is recycled to the photobioreactor process.

49. (canceled)

50. The method of claim 42, wherein the VOC is ethanol and the ethanol concentration of the condensate ranges from about 80 wt % to about 95 wt %.

51. The method of claim 42, wherein the VOC vapor stream discharged from the adsorber is recycled back to a photobioreactor process.

52. A system for recovering and concentrating a volatile organic compound (VOC) from a dilute VOC vapor stream, comprising:
a column comprising at least an adsorber, a transition, and a stripper in fluid communication;
a dilute VOC vapor stream in fluid communication with the adsorber;
a stripper gas stream in fluid communication with the stripper;
a plurality of microbeads configured to fall through the column and adsorb and desorb at least a portion of the VOC vapor;
a heat source in fluid communication with the stripper; and
a condenser configured to cool the desorbed VOC vapor and form a VOC condensate.

53. The system of claim 52, wherein the VOC is ethanol and the system further comprises a photobioreactor system producing the dilute ethanol vapor stream.

54. The system of claim 52, wherein the VOC is ethanol and the concentration of ethanol in the dilute vapor stream is about 0.04 mol % to about 1.8 mol %.

55. The system of claim 52, wherein the heat source is configured to heat the falling microbeads and adsorbed VOC vapor causing the VOC vapor to desorb, wherein the heating is done by indirect contact with the falling microbeads.

56. The system of claim 52, where in the system is configured for continuous operation.

57. The system of claim 52, wherein the transition is configured to remove at least a portion of the water before releasing the adsorbed VOC.

58. The system of claim 52, wherein the falling microbeads in the stripper operate as a moving bed and the speed of the bed corresponds to the microbeads’ residence time for efficient VOC desorption.

59. The system of claim 52, wherein the VOC is ethanol and the dilute ethanol vapor is a product of a photobioreactor process, and the stripper gas source is CO2 that is recycled back to the photobioreactor process.

60. The system of claim 52, wherein the VOC is ethanol and the ethanol concentration of the ethanol condensate ranges from about 80 wt % to about 95 wt %.

61. The system of claim 52, wherein the VOC is ethanol and the dilute ethanol vapor stream discharged from the adsorber is recycled.

62. The system of claim 52, wherein a structured packing within the column is configured such that the pressure drop is less than about 0.04 psi.
